SEQUENCE LISTING (103 sequences)

SEQ ID NO: 1 - DNA, 6075 bp, Homo sapiens
  CDS (1)..(6075)
atggccacaggaggaggtccctttgaagatggcatgaatgatcaggatttaccaaactggagtaatgagaatgttgatgacaggctcaacaatatggattggggtgcccaacagaagaaagcaaatagatcatcagaaaagaataagaaaaagtttggtgtagaaagtgataaaagagtaaccaatgatatttctccggagtcgtcaccaggagttggaaggcgaagaacaaagactccacatacgttcccacacagtagatacatgagtcagatgtctgtcccagagcaggcagaattagagaaactgaaacagcggataaacttcagtgatttagatcagagaagcattggaagtgattcccaaggtagagcaacagctgctaacaacaaacgtcagcttagtgaaaaccgaaagcccttcaactttttgcctatgcagattaatactaacaagagcaaagatgcatctacaagtcccccaaacagagaaacgattggatcagcacagtgtaaagagttgtttgcttctgctttaagtaatgacctcttgcaaaactgtcaggtgtctgaagaagatgggaggggagaacctgcaatggagagcagccagattgtaagcaggcttgttcaaattcgcgattatattactaaagctagttccatgcgggaagatcttgtagagaaaaatgagagatctgctaatgttgagcgccttactcatctaatagatcaccttaaagaacaagagaagtcatatatgaaatttcttaaaaaaatccttgccagagatcctcagcaggagcctatggaagagatagaaaatttgaagaaacaacatgatttattaaaaagaatgttacaacagcaggagcaactaagaggagctctacagggacggcaggctgcacttctagctctgcaacataaagcagacgaagctattgcagtgatggatgattctgttgttgcagaaactgcaggtagcttatctggcgtcagtatcacatctgaactaaatgaagaattgaatgacttaattcagcgttttcataatcagcttcgtgattctcagcctccagctgttccagacaatagaagacaggcagaaagtctttcattaactagggaggtttcccagagcaggaaaccatcagcttcagaacgtttacctgatgagaaagtcgaactttttagcaaaatgagagtgctacaggaaaagaaacaaaaaatggacaaattgcttggagaacttcatacacttcgagatcagcatcttaacaattcatcatcctctccacaaaggagtgtcgatcagagaagtacttcagctccctctgcttgtctaggcttggcaccggttgtcaatggagaatccaatagcctcacatcatctgttccttatcctactgcttctctagtatctcagaatgagagtgaaaacgaaggccacctcaatccatctgaaaaactccagaagttaaatgaagttcgaaagagattgaatgagctaagagaattagttcattattatgaacaaacgtcagacatgatgacagatgctgtgaatgaaaacaggaaagatgaagaaactgaagagtcagaatatgattctgagcatgaaaattccgagcctgttactaacattcgaaatccacaagtagcttccacttggaatgaagtaaatagtcatagtaatgcacagtgtgtttctaataatagagatgggcgaacagttaattctaattgtgaaattaacaacagatctgctgccaacataagggctctaaacgtgcctccttctttagattgtcgatataatagagaaggggaacaggagattcatgttgcacaaggtgaagatgatgaggaggaggaggaagaagcagaagaggagggagtcagtggagcttcattatctagtcacaggagcagtctggttgatgagcatccagaagatgctgaatttgaacagaagatcaaccgacttatggctgcaaaacagaaacttagacagttacaagatcttgttgctatggtacaggatgatgatgcagctcaaggagttatctctgccagtgcatcaaatttggatgatttctacccagcagaagaagacaccaagcaaaattcaaataacactagaggaaatgccaataaaacacagaaagatactggagtaaatgaaaaggcaagagagaaattttatgaggctaaactacagcagcaacagagagagctaaaacaattgcaggaagaaagaaagaaactgattgacattcacgagaaaattcaagcattgcaaacggcatgccctgacttacagctgtcagctgctagtgtgggtaactgtcctaccaaaaaatatatgccagctgttacttcaaccccaactgttaatcaacacgagaccagtacaagcaaatctgtttttgagcctgaagattcttcaatagtagataatgagttgtggtcagaaatgagaagacatgaaatgttgagggaggagctgcgacagagaagaaagcagcttgaagctctgatggctgaacatcagaggaggcaaggtctagctgaaactgcatctccagtggctgtgtcattgagaagtgatggatctgagaacctatgtactcctcagcaaagtagaacagaaaaaacgatggcaacttggggagggtctacccagtgtgcactagatgaagaaggagatgaagacggttacctttctgaaggaattgttcggacagatgaagaggaggaagaagagcaagatgccagttccaatgataacttttctgtgtgtccttctaacagtgtgaatcataactcctacaatcgaaaggaaactaaaaatacgtggaagaacaattgccctttttcggcagatgaaaattatcgtcctttagccaagacaaggcaacagaatatcagcatgcaacggcaagaaaaccttcgttgggtgtcagagctctcttacgtagaagagaaagaacaatggcaagaacaaatccagctaaagaaacagcttgattttagtgtcagtatttgtcagactttgatgcaagaccagcagactctatcttgtctgctacaaactcttctcacgggtccttacagtgttatgcccagcaatgttgcatctcctcaagtacacttcataatgcaccagttgaaccagtgctatactcagctaacatggcaacagaataatgttcagaggttgaaacaaatgctaaatgaacttatgcgccaacgaaatcagcatccagaaaaacctggaggcaaggaaagaggcagtagtgcatcgcaccctccttctcccagtttattttgtcctttcagctttccaacacagcctgtaaatctcttcaatatacctggatttactaacttttcatcatttgcaccaggtatgaatttcagccctttatttccttctaattttggagatttttctcagaatatctctacacccagtgaacagcagcaacccttagccagaattctttcaggaaaaacagaatatatggcttttccaaaaccttttgaaagcagttcctctattggagcagagaaaccaaggaataaaaaactgcctgaagaggaggtggaaagcagtaggacaccatggttatatgaacaagaaggtgaagtagagaaaccatttatcaagactggattttcagtgtctgtagaaaaatctacaagtagtaaccgcaaaaatcaattagatacaaacggaagaagacgccagtttgatgaagaatcactggaaagctttagcagtatgcctgatccagtagatccaacaacagtgactaaaacattcaagacaagaaaagcgtctgcacaggccagcctggcatctaaagataaaactcccaagtcaaaaagtaagaagaggaattctactcagctgaaaagcagagttaaaaacatcaggtatgaaagtgccagtatgtctagcacatgtgaaccttgcaaaagtaggaacagacattcagcccagactgaagagcctcttcaagcaaaagtattcagcagaaagaatcatgagcaactggaaaaaataataaaatgtaataggtctacagaaatatcttcagaaactgggagtgatttttccatgtttgaagctttgcaggatactatttattctgaagtagctacattaatttctcaaaatgaatctcgtccacattttcttattgaactcttccatgagctgcagctactaaacacagactacttgagacagagggctttatatgcattgcaggacatagtatccagacatatttctgagagccatgaaaaaggagaaaatgtaaagtcagtaaactctggtacttggatagcatcaaactcagaacttactcctagtgagagccttgctactactgatgatgaaacttttgagaagaactttgaaagagaaacccataaaataagtgagcaaaatgatgctgataatgctagtgtcctgtctgtatcatcaaattttgagccttttgcaacagatgatctaggtaacaccgtgattcacttagatcaagcattagccagaatgagagaatatgagcgtatgaagactgaggctgaaagtaactcaaatatgagatgcacctgcaggattattgaggatggagatggtgctggtgcaggtactacagttaataatttagaagaaactcccgttattgaaaatcgtagttcacaacaacctgtaagtgaagtttctaccatcccatgtcctagaattgatactcagcagctggaccggcaaattaaagcaattatgaaagaagtcattccttttttgaaggagcacatggatgaagtatgctcctcgcagcttctaacttcagtaaggcgcatggttttgacccttacccagcaaaatgatgagagcaaagagtttgtaaagttctttcataaacaacttggaagtatattacaggattcactggcaaaatttgctggcagaaaactgaaagactgtggagaagatcttcttgtagagatatctgaagtgttgttcaatgaattggctttctttaagcttatgcaagatttggataataatagtataactgttaaacagagatgcaaaaggaaaatagaagcaactggagtgatacaatcttgtgccaaagaggctaaaaggattcttgaagatcatggctcacctgctggagagattgatgatgaagacaaagacaaggatgaaactgaaacagttaagcagactcaaacatctgaggtgtatgatggtcccaaaaatgtaagatctgatatttctgatcaagaggaagatgaagaaagtgaaggatgtccagtgtctattaatttgtctaaagctgaaactcaggctttaactaattatggaagtggagaagatgaaaatgaggatgaagaaatggaagaatttgaagaaggccctgtggatgtccagacttccctccaggctaacactgaagctactgaagaaaatgaacatgatgaacaggtcctacaacgtgactttaaaaagacagcagaaagcaaaaatgtccattggaacgagaagccactagtaaaaatgaccaaaaataactgtcctgtgaaacccagttacctcaatatcttggaagatgagcaacctttaaatagtgctgcccataaggagtcacctcctactgttgattcaactcaacagcctaaccctttgccgttacgtttacctgaaatggaacccttagtgcctagagtcaaagaagttaaatctgctcaggaaactcctgaaagctctctggctggaagtcctgatactgaatctccagtgttagtgaatgactatgaagcagaatctggtaatataagtcaaaagtctgatgaagaagattttgtaaaagttgaagatttaccactgaaactgacaatatattcagaggcagatctaagaaagaaaatggtagaagaagaacagaaaaaccatttatctggtgaaatgtgtgaaatgcagaccgaagaattagctggaaattctgagacactaaaagaacctgaaacggtgggagcccagagtatatga

SEQ ID NO: 2 - protein, 2024 aa, Homo sapiens
MATGGGPFEDGMNDQDLPNWSNENVDDRLNNMDWGAQQKKANRSSEKNKKKFGVESDKRVTNDISPESSPGVGRRRTKTPHTFPHSRYMSQMSVPEQAELEKLKQRINFSDLDQRSIGSDSQGRATAANNKRQLSENRKPFNFLPMQINTNKSKDASTSPPNRETIGSAQCKELFASALSNDLLQNCQVSEEDGRGEPAMESSQIVSRLVQIRDYITKASSMREDLVEKNERSANVERLTHLIDHLKEQEKSYMKFLKKILARDPQQEPMEEIENLKKQHDLLKRMLQQQEQLRGALQGRQAALLALQHKADEAIAVMDDSVVAETAGSLSGVSITSELNEELNDLIQRFHNQLRDSQPPAVPDNRRQAESLSLTREVSQSRKPSASERLPDEKVELFSKMRVLQEKKQKMDKLLGELHTLRDQHLNNSSSSPQRSVDQRSTSAPSACLGLAPVVNGESNSLTSSVPYPTASLVSQNESENEGHLNPSEKLQKLNEVRKRLNELRELVHYYEQTSDMMTDAVNENRKDEETEESEYDSEHENSEPVTNIRNPQVASTWNEVNSHSNAQCVSNNRDGRTVNSNCEINNRSAANIRALNVPPSLDCRYNREGEQEIHVAQGEDDEEEEEEAEEEGVSGASLSSHRSSLVDEHPEDAEFEQKINRLMAAKQKLRQLQDLVAMVQDDDAAQGVISASASNLDDFYPAEEDTKQNSNNTRGNANKTQKDTGVNEKAREKFYEAKLQQQQRELKQLQEERKKLIDIHEKIQALQTACPDLQLSAASVGNCPTKKYMPAVTSTPTVNQHETSTSKSVFEPEDSSIVDNELWSEMRRHEMLREELRQRRKQLEALMAEHQRRQGLAETASPVAVSLRSDGSENLCTPQQSRTEKTMATWGGSTQCALDEEGDEDGYLSEGIVRTDEEEEEEQDASSNDNFSVCPSNSVNHNSYNRKETKNTWKNNCPFSADENYRPLAKTRQQNISMQRQENLRWVSELSYVEEKEQWQEQIQLKKQLDFSVSICQTLMQDQQTLSCLLQTLLTGPYSVMPSNVASPQVHFIMHQLNQCYTQLTWQQNNVQRLKQMLNELMRQRNQHPEKPGGKERGSSASHPPSPSLFCPFSFPTQPVNLFNIPGFTNFSSFAPGMNFSPLFPSNFGDFSQNISTPSEQQQPLARILSGKTEYMAFPKPFESSSSIGAEKPRNKKLPEEEVESSRTPWLYEQEGEVEKPFIKTGFSVSVEKSTSSNRKNQLDTNGRRRQFDEESLESFSSMPDPVDPTTVTKTFKTRKASAQASLASKDKTPKSKSKKRNSTQLKSRVKNIRYESASMSSTCEPCKSRNRHSAQTEEPLQAKVFSRKNHEQLEKIIKCNRSTEISSETGSDFSMFEALQDTIYSEVATLISQNESRPHFLIELFHELQLLNTDYLRQRALYALQDIVSRHISESHEKGENVKSVNSGTWIASNSELTPSESLATTDDETFEKNFERETHKISEQNDADNASVLSVSSNFEPFATDDLGNTVIHLDQALARMREYERMKTEAESNSNMRCTCRIIEDGDGAGAGTTVNNLEETPVIENRSSQQPVSEVSTIPCPRIDTQQLDRQIKAIMKEVIPFLKEHMDEVCSSQLLTSVRRMVLTLTQQNDESKEFVKFFHKQLGSILQDSLAKFAGRKLKDCGEDLLVEISEVLFNELAFFKLMQDLDNNSITVKQRCKRKIEATGVIQSCAKEAKRILEDHGSPAGEIDDEDKDKDETETVKQTQTSEVYDGPKNVRSDISDQEEDEESEGCPVSINLSKAETQALTNYGSGEDENEDEEMEEFEEGPVDVQTSLQANTEATEENEHDEQVLQRDFKKTAESKNVHWNEKPLVKMTKNNCPVKPSYLNILEDEQPLNSAAHKESPPTVDSTQQPNPLPLRLPEMEPLVPRVKEVKSAQETPESSLAGSPDTESPVLVNDYEAESGNISQKSDEEDFVKVEDLPLKLTIYSEADLRKKMVEEEQKNHLSGEMCEMQTEELAGNSETLKEPETVGAQSI

SEQ ID NO: 3 - DNA, 60 bp, Homo sapiens
acatattatttttgtagtatctcagtgtgtatttactgatgacattctgatgccacccca

SEQ ID NO: 4 - DNA, 60 bp, Homo sapiens
acatattatttttgtggtatctcagtgtgtatttactgatgacattctgatgccacccca

SEQ ID NO: 5 - DNA, 60 bp, Homo sapiens
acatattatttttgtrgtatctcagtgtgtatttactgatgacattctgatgccacccca

SEQ ID NO: 6 - DNA, 60 bp, Homo sapiens
aaatgagagatctgctaatgttgagcgccttactcatctaatagatcaccttaaagaaca

SEQ ID NO: 7 - DNA, 60 bp, Homo sapiens
aaatragagatctgctaatgttgagcgccttactcatctaatagatcaccttaaagaaca

SEQ ID NO: 8 - DNA, 60 bp, Homo sapiens
gaggctatatatatatctctgtgtcatcattctgttcttactcttttttcttgatgtcta

SEQ ID NO: 9 - DNA, 60 bp, Homo sapiens
gaggctatatatatatttctgtgtcatcattctgttcttactcttttttcttgatgtcta

SEQ ID NO: 10 - DNA, 60 bp, Homo sapiens
gaggctatatatatatytctgtgtcatcattctgttcttactcttttttcttgatgtcta

SEQ ID NO: 11 - DNA, 60 bp, Homo sapiens
ttgataattgaaaccgcaacatggattttaatagtcagatacaagtattgttggtcttat

SEQ ID NO: 12 - DNA, 60 bp, Homo sapiens
ttgataattgaaaccgcaacatggattttaatagtcaratacaagtattgttggtcttat

SEQ ID NO: 13 - DNA, 18 bp, Homo sapiens
aacaatatggtatgattc

SEQ ID NO: 14 - DNA, 18 bp, Homo sapiens
tttttaaaggattggggt

SEQ ID NO: 15 - DNA, 18 bp, Homo sapiens
ttagatcaggtttgtgaa

SEQ ID NO: 16 - DNA, 18 bp, Homo sapiens
catatatagagaagcatt

SEQ ID NO: 17 - DNA, 18 bp, Homo sapiens
agcagccaggtgataacg

SEQ ID NO: 18 - DNA, 18 bp, Homo sapiens
cccctgcagattgtaagc

SEQ ID NO: 19 - DNA, 18 bp, Homo sapiens
aaaatccttgtaagtatt

SEQ ID NO: 20 - DNA, 18 bp, Homo sapiens
attttccaggccagagat

SEQ ID NO: 21 - DNA, 18 bp, Homo sapiens
atgattctggtatgtcac

SEQ ID NO: 22 - DNA, 18 bp, Homo sapiens
tctttccagttgttgcag

SEQ ID NO: 23 - DNA, 18 bp, Homo sapiens
gattctcaggtaacctag

SEQ ID NO: 24 - DNA, 18 bp, Homo sapiens
tttaataagcctccagct

SEQ ID NO: 25 - DNA, 18 bp, Homo sapiens
attcatcatgtgagtaaa

SEQ ID NO: 26 - DNA, 18 bp, Homo sapiens
cttcttcagcctctccac

SEQ ID NO: 27 - DNA, 18 bp, Homo sapiens
aaaactccagtaaaacat

SEQ ID NO: 28 - DNA, 18 bp, Homo sapiens
gtttatcaggaagttaaa

SEQ ID NO: 29 - DNA, 18 bp, Homo sapiens
taacattcggtaagaact

SEQ ID NO: 30 - DNA, 18 bp, Homo sapiens
ttcttctagaaatccaca

SEQ ID NO: 31 - DNA, 18 bp, Homo sapiens
cttctttaggtatgactg

SEQ ID NO: 32 - DNA, 18 bp, Homo sapiens
tttcagcagattgtcgat

SEQ ID NO: 33 - DNA, 18 bp, Homo sapiens
atggtacaggtaaatatt

SEQ ID NO: 34 - DNA, 18 bp, Homo sapiens
gctcttcaggatgatgat

SEQ ID NO: 35 - DNA, 18 bp, Homo sapiens
aaaggcaaggtatgttaa

SEQ ID NO: 36 - DNA, 18 bp, Homo sapiens
ttattgtagagagaaatt

SEQ ID NO: 37 - DNA, 18 bp, Homo sapiens
gacttacaggtaattatg

SEQ ID NO: 38 - DNA, 18 bp, Homo sapiens
cctttttagctgtcagct

SEQ ID NO: 39 - DNA, 18 bp, Homo sapiens
gataatgaggtattgtaa

SEQ ID NO: 40 - DNA, 18 bp, Homo sapiens
aaaaaatagttgtggtca

SEQ ID NO: 41 - DNA, 18 bp, Homo sapiens
aacagaaaagtaagagag

SEQ ID NO: 42 - DNA, 18 bp, Homo sapiens
aatcactagaacgatggc

SEQ ID NO: 43 - DNA, 18 bp, Homo sapiens
taaaaataggttagtttc

SEQ ID NO: 44 - DNA, 18 bp, Homo sapiens
cttggttaggtggaagaa

SEQ ID NO: 45 - DNA, 18 bp, Homo sapiens
gaccagcaggtaaaattt

SEQ ID NO: 46 - DNA, 18 bp, Homo sapiens
tttcaatagactctatct

SEQ ID NO: 47 - DNA, 18 bp, Homo sapiens
tgttcagaggtaatctgt

SEQ ID NO: 48 - DNA, 18 bp, Homo sapiens
tctttgtaggttgaaaca

SEQ ID NO: 49 - DNA, 18 bp, Homo sapiens
ttgcaccaggtaggtgac

SEQ ID NO: 50 - DNA, 18 bp, Homo sapiens
actgattaggtatgaatt

SEQ ID NO: 51 - DNA, 18 bp, Homo sapiens
gaaaccaaggtacctgat

SEQ ID NO: 52 - DNA, 18 bp, Homo sapiens
attggtaaggaataaaaa

SEQ ID NO: 53 - DNA, 18 bp, Homo sapiens
aaaacatcagtaagtgtt

SEQ ID NO: 54 - DNA, 18 bp, Homo sapiens
ttactcaagggtatgaaa

SEQ ID NO: 55 - DNA, 18 bp, Homo sapiens
tatcttcaggtatgttct

SEQ ID NO: 56 - DNA, 18 bp, Homo sapiens
gtattccagaaactggga

SEQ ID NO: 57 - DNA, 19 bp, Homo sapiens
gcattgcaggtatctggta

SEQ ID NO: 58 - DNA, 18 bp, Homo sapiens
cttatttaggacatagta

SEQ ID NO: 59 - DNA, 18 bp, Homo sapiens
actgatgatgtaagctga

SEQ ID NO: 60 - DNA, 18 bp, Homo sapiens
gtaacttaggaaactttt

SEQ ID NO: 61 - DNA, 18 bp, Homo sapiens
atgatctaggtaagcaga

SEQ ID NO: 62 - DNA, 17 bp, Homo sapiens
caaagtaggtaacaccg

SEQ ID NO: 63 - DNA, 18 bp, Homo sapiens
atttagaaggtatatatt

SEQ ID NO: 64 - DNA, 18 bp, Homo sapiens
aaaaattagaaactcccg

SEQ ID NO: 65 - DNA, 18 bp, Homo sapiens
tttttgaaggtaagcaat

SEQ ID NO: 66 - DNA, 18 bp, Homo sapiens
ttttctcaggagcacatg

SEQ ID NO: 67 - DNA, 18 bp, Homo sapiens
atattacaggtaagagtt

SEQ ID NO: 68 - DNA, 18 bp, Homo sapiens
tttgaataggattcactg

SEQ ID NO: 69 - DNA, 18 bp, Homo sapiens
gccaaagaggtaaataac

SEQ ID NO: 70 - DNA, 18 bp, Homo sapiens
gttttaaaggctaaaagg

SEQ ID NO: 71 - DNA, 18 bp, Homo sapiens
gaagacaaagtatgtgct

SEQ ID NO: 72 - DNA, 18 bp, Homo sapiens
tgtgtctaggacaaggat

SEQ ID NO: 73 - DNA, 18 bp, Homo sapiens
tgtctattagtaagttta

SEQ ID NO: 74 - DNA, 18 bp, Homo sapiens
tcaacttagatttgtcta

SEQ ID NO: 75 - DNA, 18 bp, Homo sapiens
gatgaacaggtattcccg

SEQ ID NO: 76 - DNA, 18 bp, Homo sapiens
cccttctaggtcctacaa

SEQ ID NO: 77 - DNA, 18 bp, Homo sapiens
ctagtaaaagtaagaaat

SEQ ID NO: 78 - DNA, 18 bp, Homo sapiens
tgatgacagatgaccaaa

SEQ ID NO: 79 - DNA, 18 bp, Homo sapiens
aatgactatgtatgtatc

SEQ ID NO: 80 - DNA, 18 bp, Homo sapiens
acattacaggaagcagaa

SEQ ID NO: 81 - DNA, 18 bp, Homo sapiens
tattcagaggtatttagc

SEQ ID NO: 82 - DNA, 18 bp, Homo sapiens
attccaaaggcagatcta

SEQ ID NO: 83 - DNA, 18 bp, Homo sapiens
aagaacctggtaagagtt

SEQ ID NO: 84 - DNA, 18 bp, Homo sapiens
gtttttcagaaacggtgg

SEQ ID NO: 85 - DNA, 21 bp, Artificial sequence
  Primer
aattccccaaacaaaacaaca

SEQ ID NO: 86 - DNA, 20 bp, Artificial sequence
  Primer
aggctatcctttcctcagca

SEQ ID NO: 87 - DNA, 20 bp, Artificial sequence
  Primer
atgttcagccaccatcgtct

SEQ ID NO: 88 - DNA, 20 bp, Artificial sequence
  Primer
cagtgtcgctggaaagttga

SEQ ID NO: 89 - DNA, 21 bp, Artificial sequence
  Primer
tcccgaagtgctaggattaca

SEQ ID NO: 90 - DNA, 20 bp, Artificial sequence
  Primer
gctcagcaggaagaggaatg

SEQ ID NO: 91 - DNA, 20 bp, Artificial sequence
  Primer
agaggccaggcacaaaagta

SEQ ID NO: 92 - DNA, 20 bp, Artificial sequence
  Primer
aacattccagcatcccaaag

SEQ ID NO: 93 - DNA, 20 bp, Artificial sequence
  Primer
gacccactgccacactcttt

SEQ ID NO: 94 - DNA, 20 bp, Artificial sequence
  Primer
ggagtgcggcatgaaattat

SEQ ID NO: 95 - DNA, 26 bp, Artificial sequence
  Primer
atatgtatacaatgtgtatctgtatc

SEQ ID NO: 96 - DNA, 22 bp, Artificial sequence
  Primer
ccttttagttcccattcccatt

SEQ ID NO: 97 - DNA, 20 bp, Artificial sequence
  Primer
tgatgcaggagaattgcttg

SEQ ID NO: 98 - DNA, 20 bp, Artificial sequence
  Primer
cctacttggctgggattctg

SEQ ID NO: 99 - DNA, 130 bp, Homo sapiens
gtttttcagaaacggtgggagcccagagtatatgagatgtcttcagaggctcatctaactctgtccttacatactcaatgcatatatgaaaacaatactaaataaacatctgatctgtataaaaatgtaa

SEQ ID NO: 100 - DNA, 43 bp, Artificial sequence
  Primer
ggataacaatttcacacaggccaagtgtctttggttatcttcg

SEQ ID NO: 101 - DNA, 39 bp, Artificial sequence
  Primer
cacgacgttgtaaaacgacagtccgaacatcctcctcct

SEQ ID NO: 102 - DNA, 36 bp, Artificial sequence
  Primer
ggataacaatttcacacaggtgagccattgattatg

SEQ ID NO: 103 - DNA, 36 bp, Artificial sequence
  Primer
cacgacgttgtaaaacgacagttgtccctgcaacct

(19) United States
(12) Patent Application Publication
(10) Pub. No.: US 2005/0142552 A1
(43) Pub. Date: Jun. 30, 2005

(54) Susceptibility locus for schizophrenia

(76) Inventor: Hugh Malcolm Douglas Gurling, London (GB)

Correspondence Address: NIXON & VANDERHYE, PC, 1100 N GLEBE ROAD, 8TH FLOOR, ARLINGTON, VA, 22201-4714, US

(21) Appl. No.: 10/498,794

(22) PCT Filed: Dec. 12, 2002
(86) PCT No.: PCT/GB02/05630

(30) Foreign Application Priority Data
Dec. 12, 2001 (GB) 0129758.9

(51) Int. Cl. C12Q 1/68; C07H 21/04

(52) U.S. Cl. 435/6; 536/24.3

(57) ABSTRACT

Provided are methods for determining the susceptibility of an individual to a neuropsychiatric disorder, or a method of diagnosis or prognosis of the neuropsychiatric disorder (particularly schizophrenia) the methods comprising use of a pericentriolar material 1 (PCM1) marker which is located in the chromosomal region 8p21-22, for example a marker within the PCM1 gene locus or within 1000 kb of it. The invention also provides novel markers, and related materials and methods of detecting them, and identifying further molecules for use in therapy and diagnosis.

### TECHNICAL FIELD

This invention relates to the identification of chromosomal regions on chromosome 8 linked to genetic sequences which affect susceptibility to schizophrenia.

### BACKGROUND OF INVENTION

The schizophrenias are a collection of psychotic psychiatric disorders with a lifetime prevalence of approximately 0.85% in the general population. It is one of the most prevalent and potentially devastating of the neuropsychiatric disorders and is characterised by episodes of auditory hallucinations, delusions, thought disorder, inappropriate affect, bizarre behaviour and cognitive abnormalities.

Currently, individuals are typically evaluated for schizophrenia using the criteria set forth in the most current version of the International Classification of diseases or the American Psychiatric Association's Diagnostic and Statistical Manual of Mental Disorders (DSM). Some of the available drug treatments are effective in only approximately a third of individuals diagnosed with schizophrenia, a third respond partially and a third respond poorly. It has not so far been possible to predict which drug treatments will be effective in a particular schizophrenia patient.

The existence of a genetic component for schizophrenia is supported by twin studies and genetic linkage studies (Gurling, 1996, The Genetics of the Schizophrenias, In: Genetics of Mental Disorders Part II: Clinical Issues, Eds: Papadimitriou, G. N., Mendlewicz, J. Balliere's Clinical Psychiatry, International Practice and Research 2:15-46).

Mapping genes for common diseases such as schizophrenia is complicated by the variable definition of schizophrenia phenotypes, by aetiologic heterogeneity, and by uncertainty about the mode of genetic transmission of the disease trait. With neuropsychiatric disorders there is ambiguity in distinguishing individuals who carry the affected genotype from those who are genetically unaffected.

Thus, one of the greatest difficulties facing psychiatric geneticists is uncertainty regarding the validity of phenotype designations, since clinical diagnoses are based solely on clinical observation and subjective reports.

### DISCLOSURE OF THE INVENTION

The present inventors have identified a region on chromosome 8 that harbours a susceptibility locus for schizophrenia. In particular, they have identified a gene (PCM1) which appears to have a role in schizophrenia.

Previously, linkage studies have indicated that a region of chromosome 8 (8p21-22) is linked to schizophrenia (Kendler, et al 1996; Levinson et al.,1996). For example, a marker D8S261 was shown to be located in this region. However, the results from such linkage analysis are only able to localise the disease to around 30 million to 60 million base pairs. Such linkage results span a region which is far too broad to represent a single gene. Moreover, it is not predictable from such linkage analysis where in the large region, the schizophrenia susceptibility gene lies, nor to be able to identify a single gene by positional cloning or candidate gene analysis within such a large region.

The present inventors employed linkage disequilibrium mapping and allelic association studies to identify the region of chromosome 8p21-22 which is involved in schizophrenia.

In the first stage, the inventors searched for di, tri and tetra CA/GT nucleotide repeat motifs and identified seven novel polymorphisms, or microsatellite marker loci, (D8S2612; D8S2613; D8S2614; D8S2615; D8S2616; D8S2617; D8S2618). These all localised to chromosome 8p21.3. Allelic association with schizophrenia was demonstrated for two of these (D8S2615; D8S2616) in studies with 137 schizophrenia patients and 300 ethnically matched controls, in a UK population.

In addition, the inventors demonstrated strong allelic association with a known microsatellite marker (D8S261).

These markers were shown by the inventors to be localised to the chromosome region 8p21.3. D8S261, D8S2615 and D8S2616 were localised to the PCM1 gene

D8S2612; D8S2613; D8S2614; D8S2617; D8S2618 were localised as being less than one megabase from the first marker which was shown to be in allelic association with DS261.

The present inventors then searched for polymorphisms within the PCM1 gene. After identifying the intron-exon boundaries, they carried out automated bi-directional sequencing of PCR-amplified exons and of 100 bp of intron sequence either side of the exons. This analysis was carried out on 19 schizophrenia cases from the sample used in the association study, 26 cases from British multiply-affected schizophrenia families, and 10 healthy controls. In order to increase the chances of detecting a polymorphism in linkage disequilibrium with schizophrenia, affected individuals who carried the polymorphism in D8S2616, D8S2615, or D8S261 associated with schizophrenia were selected.

The inventors screened exons 4 and 5 because of their finding that D82616, which is located at the boundary of these two exons, has strong allelic association with schizophrenia.

As a result, the inventors identified single nucleotide polymorphisms in the following locations: position 80254 in the intronic sequence 3′ to exon 4; position 80123 in exon 4; position 87366 in the intronic sequence 5′ of exon 5; position 87507 in the intronic sequence 5′ to exon 5.

These results have major implications for methods of treatment, methods of diagnosis or prognosis, methods of identifying compounds for use in methods of treatment, prognosis or diagnosis, providing compounds for use in such methods of treatment, prognosis or diagnosis.

For the purposes of this application, the various aspects are discussed with particular reference to the PCM1 gene. However, it would be clear to the skilled person that, should another gene within the region identified by the inventors be found to be a relevant gene, that the various aspects of the invention would apply equally to that gene.

Where the term PCM1 marker is used this is taken as meaning a marker within an intron or exon of the PCM1 gene, or in which is in allelic association or linkage disequilibrium with the PCM1 gene.

The inventor's have found the region surrounding the PCM1 gene to have a low evolutionary rate of recombination. Therefore the distance over which linkage disequilibrium may be found is relatively high. Accordingly a PCM1 polymorphic marker may be within 3000 kb (either side) of the PCM1 gene, preferably within 1000 kb of the PCM1 gene, more preferably within 500 kb of the PCM1 gene, more preferably within 100 kb of the PCM1 gene, more preferably within 50 kb of the PCM1 gene, most preferably within 10 kb of the PCM1 gene.

In a first aspect of the present invention there is disclosed a method for assessing the susceptibility of an individual for schizophrenia, or a method of diagnosis or prognosis of schizophrenia, which method comprises (i) obtaining a sample of genomic DNA from an individual (which sample may be all or part of the material to be analysed), (ii) using a PCM1 marker located in the chromosomal region 8p21.3 to assess the susceptibility or make said diagnosis or prognosis.

The preferred PCM1 markers may be allelic or other polymorphic markers as defined above. Preferably these markers are within the PCM1 gene.

Preferred PCM1 markers are D8S2612; D8S2613; D8S2614; D8S2615; D8S2616; D8S2617; D8S2618, D8S261. More preferred are D8S2615 and D8S2616, and D8S261, most preferred are D8S2615 and D8S2616. The location of these markers is described elsewhere herein.

It is to be noted that, while allelic association was shown by the present inventors for D8S2615 D8S2616 and D8S261 in a UK population, these and the remaining PCM1 markers; D8S2612, D8S2613, D8S2614, D8S2617, and D8S2618 may also provide useful diagnostic markers in other (for example, non-UK) populations.

Alternatively, or additionally single nucleotide polymorphisms (SNP), as described herein, may be used as markers. These include: the SNP at position 80254 of the AB020866 genomic clone; the SNP at position 80123 of the AB020866 genomic clone; the SNP at position 87366 of the AB020866 genomic clone; and the SNP at position 87507 of the AB020866 genomic clone. Further details of these SNPs are given elsewhere herein.

In further aspects, the present invention discloses the use of the PCM1 gene, or the polypeptide encoded by the PCM1 gene, in a method for identifying the susceptibility of an individual for schizophrenia, or in a method of diagnosis or prognosis of schizophrenia.

The PCM1 gene has been sequenced and the wild-type coding and regulatory sequences can be found in clones AB020866 and AB020867 of the contig NT_{—}000501 [Genome Channel:http//genome.ornl.gov/GC/cgi-bin/GCKSearchForm.cgi]. For reference, the mRNA sequence of the longest open reading frame is shown in FIG. 1 for the wild type.

Sequence for the 8p21.3 region is published in the ENTREZ database in clone AB020866 and flanking clones.

Where the various aspects of the invention use, or involve, a polypeptide encoded by the PCM1 gene, it is preferred that the polypeptide is that shown in FIG. 1.

In a further aspect the present invention discloses a method for identifying the susceptibility of an individual for schizophrenia, or a method of diagnosis or prognosis of schizophrenia, wherein said method comprises: obtaining a nucleic acid sample from an individual; and determining in that sample, the presence or absence of mutations or polymorphisms in the PCM1 gene.

A corresponding method may comprise: obtaining a nucleic acid or protein sample from an individual; and determining the level of expression from the PCM1 gene.

Where a nucleic acid sample is used, the level of expression may be determined by measuring the amount of mRNA. Where a protein sample is used, the level of PCM1 gene expression may be determined by measuring the amount of PCM1 polypeptide product.

Another aspect of the present invention is a method for identifying or isolating genetic loci associated with susceptibility to schizophrenia comprising screening genomic libraries with genetic sequence derived from PCM1 polymorphic markers located in the chromosomal region 8p21.3 and identifying open reading frames in regions adjacent to said genetic sequence. The preferred markers are those indicated above.

A region which is described as ‘adjacent’ to a genetic sequence may be within about 3000 kb of the marker, preferably within about 1000 kb, within about 500 kb away, and more preferably within about 100 kb, more preferably within 50 kb, more preferably within 10 kb of the marker.

For example, an open reading frame which is adjacent the PCM1 gene may be within about 3000 kb of the marker (since the recombination frequency is quite low, as discussed earlier), preferably within about 1000 kb, within about 500 kb away, and more preferably within about 100 kb, more preferably within 50 kb, more preferably within 10 kb of the marker.

Another aspect of the present invention is a method for mapping loci which affect susceptibility to schizophrenia by comparing a genomic region containing a particular allele of a PCM1 polymorphic marker located in the chromosomal region 8p21.3 with a genomic region containing a different allele of the same marker. The preferred markers are those indicated above.

Where the present invention relates to the analysis of nucleic acid or protein of an individual, such an individual may be one who has schizophrenia, is considered at risk from schizophrenia (e.g. by having a sibling with and/or family history of schizophrenia), or may be symptomless. The sample from the individual may be prepared from any convenient sample, for example from blood or skin tissue. A sample obtained from an individual may be analysed according to methods of the present invention. Methods of the present invention may therefore include providing a sample of nucleic acid, or protein obtained from an individual.

In a further aspect the invention provides a nucleic acid molecule comprising the PCM1 gene for use in the treatment of schizophrenia.

In a further aspect the invention provides the use of the PCM1 gene in the manufacture of a medicament for the treatment of schizophrenia.

In a further aspect the invention provides a method of treatment for schizophrenia comprising administering to a patient a nucleic acid molecule having the sequence of the PCM1 gene. The nucleic acid molecule may be within a cell, and the cell containing the nucleic acid molecule may be administered to the patient.

In a further aspect the invention provides the polypeptide encoded by the PCM1 gene for use in the treatment of schizophrenia.

In a further aspect the invention provides the use of a polypeptide encoded by the PCM1 gene in the manufacture of a medicament for the treatment of schizophrenia.

In a further aspect the invention provides a method of treatment for schizophrenia comprising administering to a patient a polypeptide encoded by the nucleic acid molecule having the sequence of the PCM1 gene.

In a further aspect the invention provides a method of treatment of schizophrenia comprising administering to a patient a substance which modulates expression from the PCM1 gene, or administering a compound which modulates the level of activity of the PCM1 gene product.

In a further aspect the invention provides a method of identifying a molecule for use in the diagnosis, prognosis or treatment of schizophrenia.

Accordingly, a method of identifying a molecule for use in the diagnosis, prognosis or treatment of schizophrenia may comprise:
admixing a test substance with a nucleic acid molecule comprising the PCM1 gene; and measuring the level of expression from that nucleic acid molecule. As discussed in more detail later, such a method is typically performed using an expression system which includes a nucleic acid molecule comprising the PCM1 gene. The level of expression can be measured by measuring the amount of PCM1 mRNA or PCM1 polypeptide product, or of a reporter gene which is linked to the PCM1 gene (or portion thereof, e.g promoter) in the expression system.

For example, for identification of molecules for treatment, therapeutic molecules may be identified, these molecules may interact with PCM1 or molecules that interact with other proteins that interact with PCM1, such that expression of PCM1 containing the schizophrenia associated polymorphism is reduced or so that proteins which counter its effect are increased in expression. For example, after expression or overexpression, protein isolation and crystallisation of the PCM1 protein can be achieved which will permit the identification of protein folds and 3D structure with X rays. Furthermore, by inference from the types of amino acid residues and their position in the PCM1 protein specific target regions of the protein may be identified as sites where new treatments for schizophrenia can be targeted.

Other methods include the “Yeast two hybrid system” and the use of transgenic mutant PCM1 animals, conditional (CRE/LOX) mutants or knockout animal models and then using the absence or relative absence or over expression of PCM1 to develop a new pharmaceutical agent such as a small molecule, peptide, oligonucleotide or antibody so that the expression of too much, too little or of abnormal mutant PCM1 protein is ameliorated. Further, the expression of PCM1 protein in cells grown in an in vivo, ex vivo or in vitro method enables therapeutic drug candidate molecules to be tested to detect an effect on the expression of normal and abnormal mutant PCM1 proteins and proteins that interact with PCM1 so that a new therapeutic agent can be created. Further description of such methods is given later.

In a further aspect the invention provides a method of identifying a molecule for use in the diagnosis, prognosis or treatment of schizophrenia, which method comprises:
admixing a test substance with a polypeptide encoded by a nucleic acid molecule comprising the PCM1 gene; and measuring the level of activity of the polypeptide. Such a method may be carried out using a sample of isolated polypeptide, or may be carried out using a nucleic acid molecule comprising the PCM1 gene which is contained within an expression system, such that the polypeptide is expressed from the nucleic acid.

In a further aspect the invention provides a method of identifying a molecule for use in the diagnosis, prognosis or treatment of schizophrenia, which method comprises:
admixing a test substance with a nucleic acid molecule comprising the PCM1 gene, or with a polypeptide encoded by a nucleic acid molecule comprising the PCM1 gene; and determining the binding of the test substance to the polypeptide. Such a method may be carried out using a nucleic acid molecule comprising the PCM1 gene which is contained within an expression system.

Further methods of identifying molecules for use in a method of diagnosis or prognosis of schizophrenia include methods of identifying substances which bind to the PCM1 nucleic acid sequence or to the PCM1 polypeptide product, and methods of identifying substances which inhibit the interaction of the PCM1 gene or PCM1 gene product with its “binding partners”.

In any of the above methods, the results may be compared with results from a control sample, in which no test substance was included.

Substances identified by the above methods (which may include novel substances) therefore represent further aspects of the invention.

In further aspects the invention provides an isolated nucleic acid which is a polymorphic variant of the nucleic acid sequence having the sequence shown in FIG. 1, such a sequence may have the polymorphic variation shown in any one of FIG. 2, 3, 4 or 5, or a combination of these. The invention further provides polypeptides encoded by these sequences where these differ from that encoded by FIG. 1. These nucleic acids have been shown by the inventors to have a role in schizophrenia.

It should be noted that The chromosome 8p21-22 PCM1 subtype of schizophrenia can also manifest itself as other psychiatric disorders. For example Kendler et al 1988 (Kendler, K. S., Gruenberg, A. M. & Tsuang, M. T. (1988) A family study of the subtypes of schizophrenia. Am J Psychiatry, 145, 57-62.) found that different subtypes of schizophrenia, schizoaffective disorder and schizotypal disorder appeared to share the same genetic aetiology within families where schizophrenia is present. In addition subtypes of common psychiatric disorders can also share the same genetic aetiology as schizophrenia as shown by Tsuang et al 1991 (Tsuang, M. T. (1991) Morbidity risks of schizophrenia and affective disorders among first-degree relatives of patients with schizoaffective disorders. Br J Psychiatry, 158, 165-170.8p21-22).

Although uncommon, subtypes of neuroses such as affective disorder, obsessive compulsive disorder, phobias and anxiety states can also be caused by the same genetic aetiology as schizophrenia. These disorders are sometimes diagnosed as “pseudoneurotic schizophrenia”. Thus where the term “schizophrenia” is used herein, it will be understood that it encompasses also these related disorders, and that the various aspects of the invention also apply to other neuropsychiatric disorders relating to the PCM1 gene, such as the related neuropsychiatric disorders, including delusional disorders, reality distortion syndrome, psychomotor retardation syndrome, confusion syndrome, paraphrenia, paranoid psychosis, schizotypal disorder, schizoaffective disorder, schizoaffective schizophrenia, psychogenic psychosis, catatonia, periodic schizophrenia, cycloid personality disorder, schizophrenia related affective disorders and subtypes of unipolar affective disorder, attention deficit disorder and magical thinking.

Various aspects of the invention will now be described in more detail.

Diagnostic Methods

The methods of the invention may use a variety of techniques and for the diagnosis or prognosis of schizophrenia, or for identification of subjects having a predisposition to, or susceptibility for, schizophrenia.

The methods described herein may be performed, for example, by utilizing pre-packaged diagnostic kits comprising at least one nucleic acid comprising the PCM1 gene, or the PCM1 polypeptide product, or comprising an antibody specific for PCM1, which may be used e.g., in clinical settings, in a method of diagnosis, or prognosis of schizophrenia. Accordingly, such a kit represents a further aspect of the invention.

Examples of techniques which may be used in the methods of the invention are described below.

Detection of PCM1 Nucleic Acid Molecules

A variety of methods can be employed to screen for the presence of PCM1 mutations and to detect and/or assay levels of PCM1 nucleic acid sequences.

Mutations within the PCM1 gene can be detected by utilizing a number of techniques. Nucleic acid from any nucleated cell can be used as the starting point for such assay techniques, and may be isolated according to standard nucleic acid preparation procedures that are well known to those of skill in the art.

PCM1 nucleic acid sequences may be used in hybridization or amplification assays of biological samples to detect abnormalities involving PCM1 gene structure, including point mutations, insertions, deletions, inversions, translocations and chromosomal rearrangements. Such assays may include, but are not limited to, Southern analyses, single-stranded conformational polymorphism analyses (SSCP), and PCR analyses.

Mutations and polymorphisms in the gene may be detected using DNA (or RNA) sequence analysis, using techniques which are standard in the art. Examples of suitable techniques may be found in Sambrook et al., 1987.

Diagnostic methods for the detection of PCM1 gene-specific mutations can involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described elsewhere herein, under conditions favorable for the specific annealing of these reagents to their complementary sequences within the PCM1 gene. Preferably, the lengths of these nucleic acid reagents are at least 15 to 30 nucleotides.

After incubation, all non-annealed nucleic acids are removed from the nucleic acid:PCM1 molecule hybrid. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled PCM1 nucleic acid reagents is accomplished using standard techniques well-known to those in the art. The PCM1 gene sequences to which the nucleic acid reagents have annealed can be compared to the annealing pattern expected from a normal PCM1 gene sequence in order to determine whether a PCM1 gene mutation is present.

Alternative diagnostic methods for the detection of PCM1 gene specific nucleic acid molecules, in patient samples or other appropriate cell sources, may involve their amplification, e.g., by PCR (the experimental embodiment set forth in Mullis, 1987, U.S. Pat. No. 4,683,202), followed by the detection of the amplified molecules using techniques well known to those of skill in the art. The resulting amplified sequences can be compared to those that would be expected if the nucleic acid being amplified contained only normal copies of the PCM1 gene in order to determine whether a PCM1 gene mutation exists.

Additionally, well-known genotyping techniques can be performed to identify individuals carrying PCM1 gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms, which can be utilized for the identification of PCM1 gene mutations, have been described that capitalize on the presence of variable numbers of short, tandemly repeated DNA sequences between the restriction enzyme sites. For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers that are so closely spaced exhibit a high frequency co-inheritance, and are extremely useful in the identification of genetic mutations, such as, for example, mutations within the PCM1 gene, and the diagnosis of diseases and disorders related to PCM1 mutations.

Also, Caskey et al. (U.S. Pat. No. 5,364,759) describe a DNA profiling assay for detecting short tri and tetra nucleotide repeat sequences. The process includes extracting the DNA of interest, such as the PCM1 gene, amplifying the extracted DNA, and labelling the repeat sequences to form a genotypic map of the individual's DNA.

The level of PCM1 gene expression may be assayed. For example, RNA from a cell type or tissue known, or suspected, to express the PCM1 gene, such as brain, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the PCM1 gene. Such analyses may reveal both quantitative and qualitative aspects of the expression pattern of the PCM1 gene, including activation or inactivation of PCM1 gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the PCM1 gene nucleic acid reagents described herein. The preferred lengths of such nucleic acid reagents are at least 9-40 nucleotides, preferably 15 to 30, more preferably 19-28 nucleotides in length.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such PCM1 gene expression assays “in situ”, i.e., directly upon tissue sections (fixed and/or frozen) of patient tissue obtained from biopsies or resections, such that no nucleic acid purification is necessary. Nucleic acid reagents such as those described herein may be used as probes and/or primers for such in situ procedures (see, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the PCM1 gene.

(2) Detection of PCM1 Gene Products

Antibodies directed against unimpaired or mutant PCM1 gene products or conserved variants or peptide fragments thereof, which are discussed, as discussed elsewhere herein, may also be used as diagnostics and prognostics for schizophrenia. Such methods may be used to detect abnormalities in the level of PCM1 gene product synthesis or expression, or abnormalities in the structure, temporal expression, and/or physical location of PCM1 gene product. Accordingly, the use of such antibodies in methods of diagnosis or prognosis of schizophrenia, and methods of diagnosis or prognosis which use these antibodies represent further aspects of the invention.

The antibodies and immunoassay methods described below have, for example, important in vitro applications in assessing the efficacy of treatments for PCM1 disorders or neuropsychiatric disorders, such as schizophrenia. Antibodies, or fragments of antibodies, such as those described below, may be used to screen potentially therapeutic compounds in vitro to determine their effects on PCM1 gene expression and PCM1 peptide production. The compounds that have beneficial effects on an PCM1 disorder or a neuropsychiatric disorder, such as schizophrenia, can be identified, and a therapeutically effective dose determined.

In vitro immunoassays may also be used, for example, to assess the efficacy of cell-based gene therapy for schizophrenia. Antibodies directed against PCM1 peptides may be used in vitro to determine, for example, the level of PCM1 gene expression achieved in cells genetically engineered to produce PCM1 peptides. In the case of intracellular PCM1 gene products, such an assessment is done, preferably, using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those that are known, or suspected, to express the PCM1 gene. The protein isolation methods employed herein may, for example, be such as those described in Harlow and Lane (1988, “Antibodies: A Laboratory Manual”, Cold Spring Harbor Laboratory Press, Cold Spring Harbor, N.Y.). The isolated cells can be derived from cell culture or from a patient. The analysis of cells taken from culture may be a necessary step in the assessment of cells to be used as part of a cell-based gene therapy technique or, alternatively, to test the effect of compounds on the expression of the PCM1 gene.

Preferred diagnostic methods for the detection of PCM1 gene products (and therefore of diagnosis or prognosis of schizophrenia) or conserved variants or peptide fragments thereof, include immunoassays wherein the PCM1 gene products or conserved variants or peptide fragments are detected by their interaction with an anti-PCM1 gene product-specific antibody.

For example, antibodies, or fragments of antibodies, such as those described herein, useful in the present invention may be used to quantitatively or qualitatively detect the presence of PCM1 gene products or conserved variants or peptide fragments thereof. This can be accomplished, for example, by immunofluorescence techniques employing a fluorescently labeled antibody (see below,) coupled with light microscopic, flow cytometric, or fluorimetric detection. Such techniques are especially preferred for PCM1 gene products that are expressed on the cell surface.

The antibodies (or fragments thereof) useful in the present invention may, additionally, be employed histologically, as in immunofluorescence or immunoelectron microscopy, for in situ detection of PCM1 gene products or conserved variants or peptide fragments thereof. In situ detection may be accomplished by removing a histological specimen from a patient, and applying thereto a labelled antibody of the present invention. The antibody (or fragment) is preferably applied by overlaying the labeled antibody (or fragment) onto a biological sample. Through the use of such a procedure, it is possible to determine not only the presence of the PCM1 gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for PCM1 gene products or conserved variants or peptide fragments thereof will typically comprise incubating a sample, such as a biological fluid, a tissue extract, freshly harvested cells, or lysates of cells, that have been incubated in cell culture, in the presence of a detectably labeled antibody capable of identifying PCM1 gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled PCM1 gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-PCM1 gene product antibody may be determined according to well known methods. Those skilled in the art will be able to determine operative and optimal assay conditions for each determination by employing routine experimentation.

One of the ways in which the PCM1 gene peptide—specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2, 1-7, Microbiological Associa'es Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, α-glycerophosphate, dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect PCM1 gene peptides through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase, green fluorescent protein and aequorin.

The diagnostic and prognostic methods of the invention may be performed on a historical sample, and the determination of the presence or absence of mutations, or the determination of the alleles of the polymorphisms described herein may be performed by analyzing DNA sequences which are stored on a database. Such a diagnostic method of the invention may comprise searching, for example a databank for a mutation within the PCM1 gene by comparison of the PCM1 gene with the sequence of the wild-type PCM1 gene, or by comparison with one of the PCM1 gene sequences shown in the Figures.

Methods to Identify Compounds that Modulate PCM1 Gene Activity

The following assays may be used in the methods of the invention which involve to the identification of molecules which modulate the expression from the PCM1 gene, or modulate the activity of the PCM1 gene product, or that bind to a PCM1 gene product, or that interfere with the interaction of a PCM1 gene product with intracellular proteins (“binding partners”). Therefore, methods of identifying a compound for use in the diagnosis, prognosis or treatment of schizophrenia which may be based on the following assays, represent further aspects of the invention.

Assays may be utilized that identify compounds that bind to PCM1 gene regulatory sequences (e.g., promoter sequences; see e.g., Platt, 1994, J. Biol. Chem. 269, 28558-28562), and that may modulate the level of PCM1 gene expression. Compounds may include, but are not limited to, small organic molecules, such as ones that are able to cross the blood-brain barrier, gain entry into an appropriate cell and affect expression of the PCM1 gene or some other gene involved in a PCM1 regulatory pathway, or intracellular proteins.

Methods for the identification of such intracellular proteins are described, below. Such intracellular proteins may be involved in the control and/or regulation of mood, or may affect the level of PCM1 gene expression and/or PCM1 gene product activity. Such compounds may be used in the therapeutic treatment schizophrenia as described below.

Compounds identified by such methods (which include novel compounds) represent further aspects of the invention. Such compounds may include, but are not limited to, peptides such as, for example, soluble peptides, including but not limited to, Ig-tailed fusion peptides, and members of random peptide libraries; (see, e.g., Lam, et al., 1991, Nature 354, 82-84; Houghten, et al., 1991, Nature 354, 84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72, 767-778), antibodies (including, but not limited to, polyclonal, monoclonal, humanized, anti-idiotypic, chimeric or single chain antibodies, and FAb, F(ab′)₂ and Fab expression library fragments, and epitope-binding fragments thereof), and small organic or inorganic molecules. Such compounds may further comprise compounds, in particular drugs or members of classes or families of drugs, known to ameliorate or exacerbate the symptoms of a neuropsychiatric disorder such as schizophrenia. Such compounds include antidepressants such as lithium salts, flupenthixol, risperidone, clozapine, quetiapine, olanzapine, haloperidol, droperidol, chlorpromazine, prochloperazine, phenothiazaine derivatives, promazine, trifluopromazine, butyrophenone derivatives, thioxanthine derivatives such as chlorprothixene and dibenzodiazepines and antipsychotic antiepileptic drugs such carbamazepine, and valproic acid, reserpine. Psychotogenic drugs such as bromocriptine, apomorphine, amphetamine, methylphenidate, methylamphetamine, ketamine, Many of these drugs are used in combination.

Compounds identified via assays such as those described herein may be useful, for example, in elaborating the biological function of the PCM1 gene product, and for ameliorating PCM1 disorders or neuropsychiatric disorders, such as schizophrenia.

The use of the following methods in the identification of compounds for use in the diagnosis, prognosis, or treatment of schizophrenia represent further aspects of the invention.

(1) In vitro Screening Assays for Compounds that Bind to the PCM1 Gene Product

In vitro systems may be designed to identify compounds capable of binding the PCM1 gene products. Compounds identified may be useful, for example, in modulating the activity of unimpaired and/or mutant PCM1 gene products, may be useful in elaborating the biological function of the PCM1 gene product, may be utilized in screens for identifying compounds that disrupt normal PCM1 gene product interactions, or may in themselves disrupt such interactions.

The principle of the assays used to identify compounds that bind to the PCM1 gene product may involve preparing a reaction mixture comprising the PCM1 gene product and the test compound under conditions and for a time sufficient to allow the two components to interact and bind, thus forming a complex that can be removed and/or detected in the reaction mixture. These assays may be conducted in a variety of ways. For example, one method to conduct such an assay would involve anchoring PCM1 gene product or the test substance onto a solid phase and detecting PCM1 gene product/test compound complexes anchored on the solid phase at the end of the reaction. In one embodiment of such a method, the PCM1 gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labelled antibody specific for the previously non-immobilized component (the antibody, in turn, may be directly labeled or indirectly labeled with a labeled anti-Ig antibody)

Alternatively, a reaction can be conducted in a liquid phase, the reaction products separated from unreacted components, and complexes detected; e.g., using an immobilized antibody specific for PCM1 gene product or the test compound to anchor any complexes formed in solution, and a labeled antibody specific for the other component of the possible complex to detect anchored complexes.

(2) Assays for Intracellular Proteins that Interact with PCM1 Gene Products

Any method suitable for detecting protein-protein interactions may be employed for identifying PCM1 protein-protein interactions.

Among the traditional methods that may be employed are co-immunoprecipitation, cross-linking and co-purification through gradients or chromatographic columns. Utilizing procedures such as these allows for the identification of proteins, including intracellular proteins, that interact with PCM1 gene products. Once isolated, such a protein can be identified and can be used in conjunction with standard techniques, to identify proteins it interacts with. For example, at least a portion of the amino acid sequence of a protein that interacts with the PCM1 gene product can be ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained may be used as a guide for the generation of oligonucleotide mixtures that can be used to screen for gene sequences encoding such proteins. Screening made be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of genes that encode the a protein which interacts with an PCM1 protein. These methods include, for example, probing expression libraries with labeled PCM1 protein, using PCM1 protein in a manner similar to the well known technique of antibody probing of lambda.gt11 and lambda.gt10 libraries.

One method that detects protein interactions in vivo, the two-hybrid system, is described in detail for illustration only and not by way of limitation. One version of this system has been described (Chien, et al., 1991, Proc. Natl. Acad. Sci. USA, 88, 9578-9582) and is commercially available from Clontech (Palo Alto, Calif.)

Briefly, utilizing such a system, plasmids are constructed that encode two hybrid proteins: one consists of the DNA-binding domain of a transcription activator protein fused to the PCM1 gene product and the other consists of the transcription activator protein's activation domain fused to an unknown protein that is encoded by a cDNA that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, PCM1 gene products may be used as the bait gene product. Total genomic or cDNA sequences are fused to the DNA encoding an activation domain. This library and a plasmid encoding a hybrid of a bait PCM1 gene product fused to the DNA-binding domain are co-transformed into a yeast reporter strain, and the resulting transformants are screened for those that express the reporter gene. For example, and not by way of limitation, a bait PCM1 gene sequence, such as the open reading frame of the PCM1 gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait PCM1 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait PCM1 gene-GAL4 fusion plasmid into a yeast strain that contains a lacZ gene driven by a promoter that contains GAL4 activation sequence. A cDNA encoded protein, fused to GAL4 transcriptional activation domain, that interacts with bait PCM1 gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait PCM1 gene-interacting protein using techniques routinely practiced in the art.

(3) Assays for Compounds that Interfere with PCM1 Gene Product Macromolecule Interaction

Assays for identifying compounds which interfere with PCM1 gene product macromolecule are described below.

PCM1 gene products of the invention may, in vivo, interact with one or more macromolecules, including intracellular macromolecules, such as proteins. Such macromolecules may include, but are not limited to, nucleic acid molecules and those proteins identified via methods such as those described, above. For purposes of this discussion, the macromolecules are referred to herein as “binding partners”. Compounds that disrupt PCM1 binding in this way may be useful in regulating the activity of the PCM1 gene product, especially mutant PCM1 gene products. Such compounds may include, but are not limited to molecules such as peptides, and the like, which would be capable of gaining access to a PCM1 gene product.

The basic principle of the assay systems used to identify compounds that interfere with the interaction between the PCM1 gene product and its binding partner or partners involves preparing a reaction mixture containing the PCM1 gene product, and the binding partner under conditions and for a time sufficient to allow the two to interact and bind, thus forming a complex.

In order to test a compound for inhibitory activity, the reaction mixture may be prepared in the presence and absence of the test compound. The test compound may be initially included in the reaction mixture, or may be added at a time subsequent to the addition of PCM1 gene product and its binding partner. Control reaction mixtures are incubated without the test compound or with a placebo. The formation of any complexes between the PCM1 gene protein and the binding partner is then detected. The formation of a complex in the control reaction, but not in the reaction mixture containing the test compound, indicates that the compound interferes with the interaction of the PCM1 gene protein and the interactive binding partner.

Complex formation within reaction mixtures containing the test compound and normal PCM1 gene protein may also be compared to complex formation within reaction mixtures containing the test compound and a mutant PCM1 gene protein. This comparison may be important in those cases wherein it is desirable to identify compounds that disrupt interactions of mutant but not normal PCM1 gene proteins.

The assay for compounds that interfere with the interaction of the PCM1 gene products and binding partners can be conducted in a heterogeneous or homogeneous format. Heterogeneous assays involve anchoring either the PCM1 gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the PCM1 gene products and the binding partners, e.g., by competition, can be identified by conducting the reaction in the presence of the test substance; i.e., by adding the test substance to the reaction mixture prior to or simultaneously with the PCM1 gene protein and interactive intracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the PCM1 gene product or the interactive binding partner, is anchored onto a solid surface, while the non-anchored species is labeled, either directly or indirectly. In practice, microtitre plates are conveniently utilized. The anchored species may be immobilized by non-covalent or covalent attachments. Non-covalent attachment may be accomplished simply by coating the solid surface with a solution of the PCM1 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the PCM1 gene protein and the interactive binding partner is prepared in which either the PCM1 gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt PCM1 gene protein/binding partner interaction can be identified.

In a particular embodiment, the PCM1 gene product can be prepared for immobilization using recombinant DNA techniques described herein. For example, the PCM1 coding region can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art, e.g., such as described herein.

This antibody can be labeled with the radioactive isotope ¹²⁵I, for example, by methods routinely practiced in the art. In a heterogeneous assay, e.g., the GST-PCM1 fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the PCM1 gene protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-PCM1 gene fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the PCM1 gene product/binding partner interaction can be detected by adding the labeled antibody and measuring the radioactivity associated with the beads.

In another embodiment of the invention, these same techniques can be employed using peptide fragments that correspond to the binding domains of the PCM1 protein and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labelled peptide comprising the binding domain may remain associated with the solid material, which can be isolated and identified by amino acid sequencing. Also, once the gene coding for the segments can be engineered to express peptide fragments of the protein, which can then be tested for binding activity and purified or synthesized.

For example, and not by way of limitation, a PCM1 gene product can be anchored to a solid material as described herein by making a GST-PCM1 fusion protein and allowing it to bind to glutathione agarose beads. The interactive binding partner obtained can be labeled with a radioactive isotope, such as ³⁵S, and cleaved with a proteolytic enzyme such as trypsin. Cleavage products can then be added to the anchored GST-PCM1 fusion protein and allowed to bind. After washing away unbound peptides, labelled bound material, representing the binding partner binding domain, can be eluted, purified, and analyzed for amino acid sequence by well-known methods. Peptides so identified can be produced synthetically or fused to appropriate facilitative proteins using recombinant DNA technology.

(4) Methods for Identification of Compounds Which Modulate the Level of Expression of the PCM1 Gene

Broadly, the level of expression of the PCM1 gene may be determined by measuring the amount of PCM1 mRNA, or polypeptide product. Suitable techniques are described in the section on “Diagnostic methods”.

Alternatively, compounds which modulate the level of PCM1 gene expression may be identified using a reporter gene system. In such a system, the PCM1 nucleic acid sequence, or portion thereof (such as the promoter) is operably linked to a reporter gene.

Use of a reporter gene facilitates determination of the level of gene expression by reference to protein production. The reporter gene preferably encodes an enzyme which catalyses a reaction which produces a detectable signal, preferably a visually detectable signal, such as a coloured product. Many examples are known, including β-galactosidase and luciferase. β-galactosidase activity may be assayed by production of blue colour on substrate, the assay being by eye or by use of a spectro-photometer to measure absorbance. Fluorescence, for example that produced as a result of luciferase activity, may be quantitated using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labelled directly or indirectly using any standard technique.

Those skilled in the art are well aware of a multitude of possible reporter genes and assay techniques which may be used to determine promoter activity. Any suitable reporter/assay may be used and it should be appreciated that no particular choice is essential to or a limitation of the present invention.

(5) Assays for Identification of Compounds that Ameliorate a PCM1 Disorder or a Neuropsychiatric Disorder

Compounds, including but not limited to binding compounds identified via assay techniques such as those described, above, may be tested for the ability to ameliorate symptoms of schizophrenia or disorder of thought and/or mood, including thought disorder, and neuropsychiatric disorders including delusional disorders, paraphrenia, paranoid psychosis, schizotypal disorder, schizoaffective disorder, schizoaffective schizophrenia, psychogenic psychosis, catatonia, periodic schizophrenia, cycloid psychosis, schizoid personality disorder, paranoid personality disorder, schizophrenia related affective disorders and subtypes of unipolar affective disorder.

It should be noted that the assays described herein can identify compounds that affect PCM1 gene activity by either affecting PCM1 gene expression or by affecting the level of PCM1 gene product activity. For example, compounds may be identified that are involved in another step in the pathway in which the PCM1 gene and/or PCM1 gene product is involved and, by affecting this same pathway may modulate the effect of PCM1 on the development of a neuropsychiatric disorder such as schizophrenia. Such compounds can be used as part of a therapeutic method for the treatment of the disorder.

First, cell-based systems can be used to identify compounds that may act to ameliorate symptoms of a PCM1 disorder or a neuropsychiatric disorder, such as schizophrenia. Such cell systems can include, for example, recombinant or non-recombinant cell, such as cell lines, that express the PCM1 gene.

In utilizing such cell systems, cells that express PCM1 may be exposed to a compound suspected of exhibiting an ability to ameliorate symptoms of a PCM1 disorder or a neuropsychiatric disorder, such as schizophrenia, at a sufficient concentration and for a sufficient time to elicit such an amelioration of such symptoms in the exposed cells. After exposure, the cells can be assayed to measure alterations in the expression of the PCM1 gene, e.g., by assaying cell lysates for PCM1 mRNA transcripts (e.g., by Northern analysis) or for PCM1 gene products expressed by the cell; compounds that modulate expression of the PCM1 gene are good candidates as therapeutics. Alternatively, the cells are examined to determine whether one or more cellular phenotypes associated with an PCM1 disorder or a neuropsychiatric disorder, such as schizophrenia, has been altered to resemble a more normal or unimpaired, unaffected phenotype, or a phenotype more likely to produce a lower incidence or severity of disorder symptoms.

In addition, animal-based systems or models for a PCM1 disorder or a neuropsychiatric disorder, such as schizophrenia, which may include, for example, PCM1 mice, may be used to identify compounds capable of ameliorating symptoms of the disorder. Such animal models may be used as test substrates for the identification of drugs, pharmaceuticals, therapies and interventions that may be effective in treating such disorders. For example, animal models may be exposed to a compound suspected of exhibiting an ability to ameliorate symptoms, at a sufficient concentration and for a sufficient time to elicit such an amelioration of symptoms of a PCM1 disorder or a neuropsychiatric disorder, such as schizophrenia, in the exposed animals. The response of the animals to the exposure may be monitored by assessing the reversal of such symptoms.

With regard to intervention, any treatments that reverse any aspect of symptoms of a PCM1 disorder or a neuropsychiatric disorder, such as schizophrenia, should be considered as candidates for human therapeutic intervention in such a disorder. Dosages of test agents may be determined by deriving dose-response curves, as discussed below.

Methods of Treatment of Schizophrenia and Compounds for Use in Such Methods

Methods of treatment of schizophrenia may comprise administering compounds which modulate the expression of a mammalian PCM1 gene and/or modulate the synthesis or activity of a mammalian PCM1 gene product. In this way the administration of such compounds results in the amelioration of the symptoms of the disorder.

Such methods may comprise supplying the mammal with a nucleic acid molecule encoding an unimpaired PCM1 gene product such that an unimpaired PCM1 gene product is expressed and symptoms of the disorder are ameliorated. Such methods maybe useful where the disorder is caused by mutations in the PCM1 gene.

Alternatively, or additionally such methods may comprise supplying the mammal with a cell comprising a nucleic acid molecule that encodes an unimpaired PCM1 gene product such that the cell expresses the unimpaired PCM1 gene product and symptoms of the disorder are ameliorated.

In cases in which a loss of normal PCM1 gene product function results in the development of a schizophrenia, an increase in PCM1 gene product activity would facilitate progress towards an asymptomatic state in individuals exhibiting a deficient level of PCM1 gene expression and/or PCM1 gene product activity. Methods for enhancing the expression or synthesis of PCM1 are described below.

Symptoms of schizophrenia, may be ameliorated by administering a compound that modulates the level of PCM1 gene expression and/or PCM1 gene product activity. Such compounds for use in the treatment of schizophrenia represent further aspects of the invention.

Suitable compounds for use in such methods may include compounds, in particular drugs, reported to ameliorate or exacerbate the symptoms of a neuropsychiatric disorder, such as schizophrenia. Such compounds include antidepressants such as lithium salts, flupenthixol, risperidone, clozapine, quetiapine, olanzapine, haloperidol, droperidol, chlorpromazine, prochloperazine, phenothiazaine derivatives, promazine, trifluopromazine, butyrophenone derivatives, thioxanthine derivatives such as chlorprothixene and dibenzodiazepines and antipsychotic antiepileptic drugs such carbamazepine, and valproic acid, reserpine. Psychotogenic drugs such as LSD, bromocriptine, apomorphine, amphetamine, methylphenidate, methylamphetaime, ketamine, Many of these drugs are used in combination.

(1) Inhibitory Antisense, Ribozyme and Triple Helix Approaches

In another embodiment, symptoms of schizophrenia may be ameliorated by decreasing the level of PCM1 gene expression and/or PCM1 gene product activity by using PCM1 gene sequences in conjunction with well-known antisense, gene “knock-out,” ribozyme and/or triple helix methods to decrease the level of PCM1 gene expression. Among the compounds that may exhibit the ability to modulate the activity, expression or synthesis of the PCM1 gene, including the ability to ameliorate the symptoms of a PCM1 disorder or a neuropsychiatric disorder, such as schizophrenia, are antisense, ribozyme, and triple helix molecules. Such molecules may be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the PCM1 gene could be used in an antisense approach to inhibit translation of endogenous PCM1 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86,6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methylguanine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15, 6625-6641). The oligonucleotide is a 2′-O-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred. For example, antisense oligonucleotides having the following sequences can be utilized in accordance with the invention:

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290, 304-310), the promoter contained in the 31 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts. For example, hammerhead ribozymes may be used.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

(2) Gene Replacement Therapy

With respect to an increase in the level of normal PCM1 gene expression and/or PCM1 gene product activity, PCM1 gene nucleic acid sequences, describe above, can, for example, be utilized for the treatment of a PCM1 disorder or a neuropsychiatric disorder, such as schizophrenia. Such treatment can be administered, for example, in the form of gene replacement therapy. Specifically, one or more copies of a normal PCM1 gene or a portion of the PCM1 gene that directs the production of a PCM1 gene product exhibiting normal PCM1 gene function, may be inserted into the appropriate cells within a patient, using vectors that include, but are not limited to adenovirus, adeno-associated virus, and retrovirus vectors, in addition to other particles that introduce DNA into cells, such as liposomes.

Because the PCM1 gene is expressed in the brain, such gene replacement therapy techniques should be capable delivering PCM1 gene sequences to these cell types within patients. Thus, in one embodiment, techniques that are well known to those of skill in the art (see, e.g., PCT Publication No. WO89/10134, published Apr. 25, 1988) can be used to enable PCM1 gene sequences to cross the blood-brain barrier readily and to deliver the sequences to cells in the brain. With respect to delivery that is capable of crossing the blood-brain barrier, viral vectors such as, for example, those described above, are preferable. Also included are methods using liposomes either in vivo ex vivo or in vitro. Wherein PCM1 sense or antisense DNA is delivered to the cytoplasm and nucleus of target cells. Liposomes can deliver PCM1 sense or nonsense RNA to humans and the human brain or in mammals through intrathecal delivery either as part of a viral vector or as DNA conjugated with nuclear localizing proteins or other proteins that increase take up into the cell nucleus.

In another embodiment, techniques for delivery involve direct administration of such PCM1 gene sequences to the site of the cells in which the PCM1 gene sequences are to be expressed. Additional methods that may be utilized to increase the overall level of PCM1 gene expression and/or PCM1 gene product activity include the introduction of appropriate PCM1-expressing cells, preferably autologous cells, into a patient at positions and in numbers that are sufficient to ameliorate the symptoms of a PCM1 disorder or a neuropsychiatric disorder, such as schizophrenia. Such cells may be either recombinant or non-recombinant.

Among the cells that can be administered to increase the overall level of PCM1 gene expression in a patient are normal cells, preferably brain cells and also choroid plexus cells within the CNS which are accessible through intrathecal injections. Alternatively, cells, preferably autologous cells, can be engineered to express PCM1 gene sequences, and may then be introduced into a patient in positions appropriate for the amelioration of the symptoms of a PCM1 disorder or a neuropsychiatric disorder, such as schizophrenia. Alternately, cells that express an unimpaired PCM1 gene and that are from a MHC matched individual can be utilized, and may include, for example, brain cells. The expression of the PCM1 gene sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above, that are capable of modulating PCM1 gene product activity can be administered using standard techniques that are well known to those of skill in the art. In instances in which the compounds to be administered are to involve an interaction with brain cells, the administration techniques should include well known ones that allow for a crossing of the blood-brain barrier such as intrathecal injection and conjugation with compounds that allow transfer across the blood brain barrier.

Pharmaceutical Preparations and Methods of Administration

Compounds that are determined to affect PCM1 gene expression or gene product activity can be administered to a patient at therapeutically effective doses to treat or ameliorate schizophrenia.

A therapeutically effective dose refers to that amount of the compound sufficient to result in amelioration of symptoms of such a disorder.

(1) Effective Dose

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

(2) Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or intrathecal, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Nucleic acid Molecules

Nucleic acid molecules for use in the various aspects of the invention may be provided isolated and/or purified from their natural environment, in substantially pure or homogeneous form, or free or substantially free of other nucleic acids of the species of origin. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser. Nucleic acid according to the present invention may include cDNA, RNA and modified nucleic acids or nucleic acid analogs. Where a DNA sequence is specified, e.g. with reference to a figure, unless context requires otherwise the RNA equivalent, with U substituted for T where it occurs, is encompassed. Where a nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby by each nucleotide is capable of base pairing with its counterpart i.e. G to C, and A to T or U.

The nucleic acid may be in the form of a recombinant, and preferably replicable vector. Preferably the nucleic acid is under the control of, and operably linked to regulatory elements which are capable of directing expression from the PCM1 nucleic acid sequence.

As used herein, regulatory elements include but are not limited to, inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus,the lac system, the trp system, the TAC system, the TRC system, the CRE/LOX system, the major operator and promoter regions of phage A, the control regions of fd coat protein, the promoter for 3-phosphoglycerate kinase, the promoters of acid phosphatase, and the promoters, of the yeast α-mating factors.

Polypeptides and Proteins

Polypeptides for use according to the various aspects of the invention may be prepared by chemical synthesis, or by using recombinant technology, as is understood by the person skilled in the art.

A variety of host-expression vector systems may be utilized to express the PCM1 gene coding sequences. Such host-expression systems represent vehicles by which the coding sequences of interest may be produced and subsequently purified, but also represent cells that may, when transformed or transfected with the appropriate nucleotide coding sequences, exhibit the PCM1 gene product of the invention in situ. Suitable host-expression systems include but are not limited to microorganisms such as bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the PCM1 gene product being expressed. For example, when a large quantity of such a protein is to be produced, e.g., for the generation of pharmaceutical compositions of PCM1 protein or for raising antibodies to the PCM1 protein, vectors that direct the expression of high levels of fusion protein products that are readily purified may be desirable. Such vectors include, but are not limited, to the E. coli expression vector pUR278 (Ruther et al., 1983, EMBO J. 2, 1791), in which the PCM1 gene product coding sequence may be ligated individually into the vector in frame with the lac Z coding region so that a fusion protein is produced; pIN vectors (Inouye and Inouye, 1985, Nucleic Acids Res. 13, 3101-3109; Van Heeke and Schuster, 1989, J. Biol. Chem. 264, 5503-5509); and the like. PGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica, nuclear polyhedrosis virus (AcNPV) may be used as a vector to express foreign genes. The virus grows in Spodoptera frugiperda cells. The PCM1 gene coding sequence may be cloned individually into non-essential regions (for example the polyhedrin gene) of the virus and placed under control of an ACNPV promoter (for example the polyhedrin promoter). Successful insertion of PCM1 gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith, et al., 1983, J. Virol. 46, 584; Smith, U.S. Pat. No. 4,215,051).

In mammalian host cells, a number of viral-based expression systems may be utilized. In cases where an adenovirus is used as an expression vector, the PCM1 gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing PCM1 gene product in infected hosts. (e.g., See Logan and Shenk, 1984, Proc. Natl. Acad. Sci. USA 81, 3655-3659).

Specific initiation signals may also be required for efficient translation of inserted PCM1 gene product coding sequences. These signals include the ATG initiation codon and adjacent sequences. In cases where an entire PCM1 gene, including its own initiation codon and adjacent sequences, is inserted into the appropriate expression vector, no additional translational control signals may be needed. However, in cases where only a portion of the PCM1 gene coding sequence is inserted, exogenous translational control signals, including, perhaps, the ATG initiation codon, should be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner, et al., 1987, Methods in Enzymol. 153, 516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines that stably express the PCM1 gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the PCM1 gene product. Such engineered cell lines may be particularly useful in screening and evaluation of compounds that affect the endogenous activity of the PCM1 gene product.

A number of selection systems may be used, including but not limited to the herpes simplex virus thymidine kinase (Wigler, et al., 1977, Cell 11, 223), hypoxanthine-guanine phosphoribosyltransferase (Szybalska and Szybalski, 1962, Proc. Natl. Acad. Sci. USA 48, 2026), and adenine phosphoribosyltransferase (Lowy, et al., 1980, Cell 22, 817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77, 3567; O'Hare, et al., 1981, Proc. Natl. Acad. Sci. USA 78, 1527); gpt, which confers resistance to mycophenolic acid (Mulligan and Berg, 1981, Proc. Natl. Acad. Sci. USA 78, 2072); neo, which confers resistance to the aminoglycoside G-418 (Colberre-Garapin, et al., 1981, J. Mol. Biol. 150, 1); and hygro, which confers resistance to hygromycin (Santerre, et al., 1984, Gene 30, 147).

Alternatively, any fusion protein may be readily purified by utilizing an antibody specific for the fusion protein being expressed. For example, a system described by Janknecht, et al. allows for the ready purification of non-denatured fusion proteins expressed in human cell lines (Janknecht, et al., 1991, Proc. Natl. Acad. Sci. USA 88, 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The PCM1 gene products can also be expressed in transgenic animals. Animals of any species, including, but not limited to, mice, rats, rabbits, guinea pigs, pigs, micro-pigs, goats, sheep, and non-human primates, e.g., baboons, monkeys, and chimpanzees may be used to generate PCM1 transgenic animals. The term “transgenic,” as used herein, refers to animals expressing PCM1 gene sequences from a different species (e.g., mice expressing human PCM1 sequences), as well as animals that have been genetically engineered to overexpress endogenous (i.e., same species) PCM1 sequences or animals that have been genetically engineered to no longer express endogenous PCM1 gene sequences (i.e., “knock-out” animals), and their progeny.

Any technique known in the art may be used to introduce an PCM1 gene transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten, et al., 1985, Proc. Natl. Acad. Sci., USA 82, 6148-6152); gene targeting in embryonic stem cells (Thompson, et al., 1989, Cell 56, 313-321); electroporation of embryos (Lo, 1983, Mol. Cell. Biol. 3, 1803-1814); and sperm-mediated gene transfer (Lavitrano et al., 1989, Cell 57, 717-723) (For a review of such techniques, see Gordon, 1989, Transgenic Animals, Intl. Rev. Cytol. 115; 171-229)

Once transgenic animals have been generated, the expression of the recombinant PCM1 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include but are not limited to Northern blot analysis of tissue samples obtained from the animal, in situ hybridization analysis, and RT-PCR (reverse transcriptase PCR). Samples of PCM1 gene-expressing tissue, may also be evaluated immunocytochemically using antibodies specific for the PCM1 transgene product.

Antibodies

Antibodies capable of specifically recognising the polypeptide encoded by the PCM1 gene, or capable of specifically recognising fragments or epitopes of the polypeptide encoded by the PCM1 gene, may be used in the various aspects of the invention.

For example, such antibodies may be used in the detection of a PCM1 gene product in an biological sample and may, therefore, be utilized as part of a diagnostic or prognostic technique whereby patients may be tested for abnormal levels of PCM1 gene products, and/or for the presence of abnormal forms of such gene products.

Such antibodies may also be utilized in conjunction with, for example, compound screening schemes, as described herein, for the evaluation of the effect of test compounds on PCM1 gene product levels and/or activity.

Such antibodies may be used to inhibit abnormal PCM1 gene product activity. Thus, such antibodies may, therefore, be used in a method of treatment or therapy for schizophrenia.

For example, antibodies may be used in a gene therapy technique, described herein, e.g., to evaluate the normal and/or engineered PCM1-expressing cells prior to their introduction into the patient.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies against a PCM1 gene product, various host animals may be immunized by injection with a PCM1 gene product, or a portion thereof. Such host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and otentially useful human adjuvants such as BCG (bacille Calmette-Guerin) and Corynebacterium parvum.

Polyclonal antibodies are heterogeneous populations of antibody molecules derived from the sera of animals immunized with an antigen, such as a PCM1 gene product, or an antigenic functional derivative thereof. For the production of polyclonal antibodies, host animals such as those described above, may be immunized by injection with PCM1 gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4, 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimaeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397)

In addition, techniques have been developed for the production of humanized antibodies. (See, e.g., Queen, U.S. Pat. No. 5,585,089, which is incorporated herein by reference in its entirety.) An immunoglobuin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242, 423-426; Huston, et al., 1988, Proc. Natl. Acad. Sci. USA 85, 5879-5883; and Ward, et al., 1989, Nature 334, 544-546) can be adapted to produce single chain antibodies against PCM1 gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse, et al., 1989, Science, 246, 1275-1281) to allow rapid and easy identification of monoclonal Fab fragments with the desired specificity.

Variants and Derivatives

Where the invention relates to methods which use the PCM1 gene, or to use of the PCM1 gene, these methods (and uses) may use a variant of the PCM 1 gene. Such variants are now discussed in more detail.

A variant nucleic acid molecule shares homology with, or is identical to, all or part of the coding sequence discussed above. Generally, variants may encode, or be used to isolate or amplify nucleic acids which encode, polypeptides which are have the same activity as the polypeptide encoded by the PCM 1 nucleic acid sequence and/or which will specifically bind to an antibody raised against the polypeptide encoded by the PCM 1 nucleic acid sequence.

Variants of the present invention can be artificial nucleic acids (i.e. containing sequences which have not originated naturally) which can be prepared by the skilled person in the light of the present disclosure. Alternatively they may be novel, naturally occurring, nucleic acids, which may be isolatable using the sequences disclosed herein.

Thus a variant may be a distinctive part or fragment (however produced) corresponding to a portion of the PCM1 gene sequence. The fragments may encode particular functional parts of the polypeptide. Equally the fragments may have utility in probing for, or amplifying, the sequence provided or closely related ones.

Also included are nucleic acids which have been extended at the 3′ or 5′ terminus.

Artificial variants (derivatives) may be prepared by those skilled in the art, for instance by site directed or random mutagenesis, or by direct synthesis. The term ‘variant’ nucleic acid as used herein encompasses all of these possibilities.

Some of the aspects of the present invention relating to variants will now be discussed in more detail.

Homology (i.e. similarity or identity) may be as defined using sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 ′ for DNA; KTUP word length: 2 for proteins/6 for DNA. Homology may be at the nucleotide sequence and/or encoded amino acid sequence level. Preferably, the nucleic acid and/or amino acid sequence shares at least about 60%, or 70%, or 80% homology, most preferably at least about 90%, 95%, 96%, 97%, 98% or 99% homology with the sequence of the PCM1 gene.

Thus a variant polypeptide in accordance with the present invention may include within its sequence, a single amino acid or 2, 3, 4, 5, 6, 7, 8, or 9 changes, about 10, 15, 20, 30, 40 or 50 changes, or greater than about 50, 60, 70, 80, 90, 100, 200 changes, compared with the polypeptide encoded by the PCM1 gene. In addition to one or more changes within the amino acid sequence shown, a variant polypeptide may include additional amino acids at the C-terminus and/or N-terminus. Naturally, regarding nucleic acid variants, changes to the nucleic acid which make no difference to the encoded polypeptide (i.e. ‘degeneratively equivalent’) are included within the scope of the present invention.

Changes to a sequence, to produce a derivative, may be by one or more of addition, insertion, deletion or substitution of one or more nucleotides in the nucleic acid, leading to the addition, insertion, deletion or substitution of one or more amino acids in the encoded polypeptide.

Changes may be desirable for a number of reasons, including introducing or removing the following features: restriction endonuclease sequences; codon usage; other sites which are required for post translation modification; cleavage sites in the encoded polypeptide; motifs in the encoded polypeptide(e.g. binding sites) Leader or other targeting sequences (e.g. hydrophobic anchoring regions) may be added or removed from the expressed protein to determine its location following expression. All of these may assist in efficiently cloning and expressing an active polypeptide in recombinant form (as described below).

Other desirable mutations may be random or site directed mutagenesis in order to alter the activity (e.g. specificity) or stability of the encoded polypeptide.

Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered stability or specificity.

The invention will now be described with reference to the following non-limiting examples and Figures.

### FIGURES, TABLES

FIG. 1 shows the longest open reading frame of Homo sapiens pericentriolar material 1 (PCM1) mRNA and the translated product. The coding sequence has a length of 6075 nt and the translated product is 2024 aa long. The translation initiation codon (atg) is at position 410 while the stop codon (tga) is at position 6482 of the PCM1 mRNA.

FIG. 2 shows the LI-COR AlignIR alignment report for a novel DNA variant identified in intronic sequence 3′ to exon 4 of PCM1 (position 80254 of the AB020866 clone). The sequence of clone AB020866 was the reference genomic sequence obtained from the Entrez nucleotide database and can be seen at the first line (ex4gnm). The variation occurs at nucleotide position 80254 of the AB020866 clone (marked X). The consensus sequence obtained from AlignIR can be seen at the last line (Consensus). Heterozygotes are marked R, G homozygotes are marked G, A homozygotes are marked A.

FIG. 3 shows the LI-COR AlignIR alignement report for a novel DNA variant identified in exon 4 of PCM1 (position 80123 of the AB020866 clone). The sequence of the cDNA of PCM1 (NM_{—}006197) can be seen at the first line (ex4cDNA) while the clone AB020866 was the reference genomic sequence and can be seen at the second line (ex4gnm). Both sequences were obtained from the Entrez nucleotide database. The variation occurs at nucleotide position 1100 of the cDNA and 80123 of the AB020866 clone and is marked X. The consensus sequence obtained from AlignIR can be seen at the last line (Consensus). Heterozygotes, are shown as R, G homozygotes as G.

FIG. 4 shows the LI-COR AlignIR alignment report for a novel DNA variant identified in intronic sequence 5′ to exon 5 of PCM1 (position 87366 of the AB020866 clone). The, sequence of clone AB020866 was the reference genomic sequence obtained from the Entrez nucleotide database and can be seen at the first line (ex5gnm). The variation occurs at nucleotide position 87366 of the AB020866 clone (marked X). The consensus sequence obtained from AlignIR can be seen at the last line (Consensus). Heterozygotes are marked as Y, C homozygotes are marked as C, T homozygotes are marked as T.

FIG. 5 shows the AlignIR alignment report for a novel DNA variant identified at position 87507 of the AB020866 clone). The sequence of clone AB020866 was the reference genomic sequence obtained from the Entrez nucleotide database and can be seen at the first line (ex5gnm). The variation occurs at nucleotide position 87507 of the AB020866 clone is marked with an X. The consensus sequence obtained from AlignIR can be seen at the last line (Consensus). Heterozygotes are marked R, G homozygotes are marked G, A homozygotes are marked A.

FIG. 6 (Table 4) shows the exon-intron boundaries referred to in Example 2.

### DETAILED DESCRIPTION OF INVENTION

The studies are described that, first, define an interval of approximately 500 kb on the short arm of human chromosome 8 within which a region associated with a neuropsychiatric disorder is located and, second, identified a novel mutation within the gene referred to herein as PCM1-EX41, which lies within this region and which is involved in neuropsychiatric disorders.

### EXAMPLE 1

### Linkage Disequilibrium

Materials and Methods

Linkage Disequilibrium.

Linkage disequilibrium (LD) studies were performed using DNA from a population sample of neuropsychiatric disorder (schizophrenia) patients. The population sample and LD techniques were as described as below. The present LD study took advantage of the discovery of additional physical markers identified via the physical mapping and sequencing techniques described below.

Bacterial Artificial Chromosome (BAC) Mapping.

For physical mapping, bacterial artificial chromosomes (BACs) containing human sequences were mapped to the region being analyzed based on publicly available maps (Human genome database, Toronto 1999 and ). The BACs were then ordered and contig reconstructed by performing standard mapping with microsatellite markers and polymorphic SNP's that were discovered by HMDG and EB and.

Bacterial artificial chromosome (BAC) mapping. Sequences flanking a microsatellite polymorphism were used to screen a human BAC library. The ends of the BACs were cloned and subclones were sequenced. From one such BAC, and from raw sequence data additional microsatellites were identified. A microsatellite sequence from the sublibrary was identified by corresponding microsatellite probes. Sequences around such repeats were obtained to enable development of PCR primers for genomic DNA.

Fluoresecent in situ hybridization (FISH) was used map the new BAC to the region being analyzed.

The resulting sequences were then compared to public DNA and protein databases using BLAST algorithms (Altschul, et al., 1990, J. Mclec. Biol., 215, 403-410).

Results

Genetic regions involved in schizophrenia had previously been reported to map to portions of the short (8p) arm of human chromosome 8. Including a broad genetic region of about 60 cM between markers on most of the short arm of chromosome 8p.

Prior to attempting to identify gene sequences, studies were performed to further narrow the neuropsychiatric disorder region.

Specifically, a linkage disequilibrium (LD) analysis was performed using population samples and techniques as described above, which took advantage of the additional physical markers identified via the physical mapping and sequencing techniques described below.

In order to provide the precise order of genetic markers necessary for linkage and LD mapping, and to guide new microsatellite marker development for finer mapping, a high resolution physical map of the 8p21-22.3 candidate region was developed (Table 1). Eleven polymorphisms, seven of which are described here for the first time (see Table 2), were genotyped in a sample of 134 schizophrenia patients of UK ancestry and 316 ethnically matched normal controls.

TABLE 1
The clones that constitute the NT 000501 contig (121,0381
bases) of the human 8p21.3-p22 sequence are shown.
The position of seven novel microsatellite markers which developed
from this sequence together with two already known markers (D8S261
and AFM333th1) are shown as well as their respective clones and the
intermarker distances between the nine markers.

| | | | POSITION | INTERMARKER |
|---|---|---|---|---|
| | | | IN THE | DISTANCES |
| CLONES | SIZE (bp) | MARKERS | CLONE (bp) | (bp) |
| AB020858.1 | 100,000 | | | |
| AB020859.1 | 100,000 | | | |
| AB020860.1 | 100,000 | | | |
| AB020861.1 | 100,000 | | | |
| AB020862.1 | 100,000 | | | |
| AB020863.1 | 156,909 | D8S2618 | 14,588 | 178,562 |
| AB020864.1 | 100,000 | D8S2613 | 36,241 | 90,549 |
| AB020865.1 | 100,000 | D8S2614 | 26,790 | 73,402 |
| AB020866.1 | 100,000 | D8S2615 | 192 | 86,911 |
| | | D8S2616 | 87,103 | 21,981 |
| AB020867.1 | 100,000 | D8S261 | 9,084 | 6,150 |
| | | AFM333th1 | 15,234 | 62,533 |
| | | D8S2617 | 77,767 | 28,400 |
| AB020868.1 | 153,472 | D8S2612 | 6,167 | |

TABLE 2

Primer sequences and allele set of the novel
microsatellite repeats identified in the NT
000501 contig on chromosome 8p21.3-22

| | |
|---|---|
| D8S2612 | |
| Primers: | 5′AAT TCC CCA AAC AAA ACA ACA3′ |
| | 5′AGG CTA TCC TTT CCT CAG CA3′ |

| | | | |
|---|---|---|---|
| Alleles | Length (bp) | (CA)n | Frequency |

| | | | |
|---|---|---|---|
| 1 | 161 | 18 | 0.0112 |
| 2 | 159 | 17 | 0.0594 |
| 3 | 157 | 16 | 0.1614 |
| 4 | 155 | 15 | 0.2578 |
| 5 | 153 | 14 | 0.4283 |
| 6 | 151 | 13 | 0.0751 |
| 7 | 149 | 12 | 0.0000 |
| 8 | 147 | 11 | 0.0000 |
| 9 | 145 | 10 | 0.0067 |

| | |
|---|---|
| Heterozygosity: | 0.7147 |
| No. of chromosomes: | 892 |

| | |
|---|---|
| D8S2617 | |
| Primers: | 5′ATG TTC AGC CAC CAT CGT CT3′ |
| | 5′CAG TGT CGC TGG AAA GTT GA3′ |

| | | | |
|---|---|---|---|
| Alleles | Length (bp) | (CA)n | Frequency |

| | | | |
|---|---|---|---|
| 1 | 203 | 25 | 0.0146 |
| 2 | 201 | 24 | 0.0822 |
| 3 | 199 | 23 | 0.1216 |
| 4 | 197 | 22 | 0.0619 |
| 5 | 195 | 21 | 0.2759 |
| 6 | 193 | 20 | 0.4358 |
| 7 | 191 | 19 | 0.0068 |
| 8 | 189 | 18 | 0.0000 |
| 9 | 187 | 17 | 0.0000 |
| 10 | 185 | 16 | 0.0011 |

| | |
|---|---|
| Heterozygosity: | 0.7083 |
| No. of chromosomes: | 888 |

| | |
|---|---|
| D8S2616 | |
| Primers: | 5′TCC CGA AGT GCT AGG ATT ACA3′ |
| | 5′GCT CAG CAG GAA GAG GAA TG3′ |

| | | | |
|---|---|---|---|
| Alleles | Length (bp) | (CA)n | Frequency |

| | | | |
|---|---|---|---|
| 3 | 213 | 24 | 0.0011 |
| 4 | 211 | 23 | 0.0113 |
| 5 | 209 | 22 | 0.0317 |
| 6 | 207 | 21 | 0.0588 |
| 7 | 205 | 20 | 0.1301 |
| 8 | 203 | 19 | 0.5848 |
| 9 | 201 | 18 | 0.0532 |
| 10 | 199 | 17 | 0.1199 |
| 11 | 197 | 16 | 0.0011 |
| 12 | 195 | 15 | 0.0057 |
| 13 | 193 | 14 | 0.0000 |
| 14 | 191 | 13 | 0.0011 |
| 15 | 189 | 12 | 0.0011 |

| | |
|---|---|
| Heterozygosity: | 0.6192 |
| No. of chromosomes: | 884 |

| | |
|---|---|
| D8S2615 | |
| Primers: | 5′AGA GGC CAG GCA CAA AAG TA3′ |
| | 5′AAC ATT CCA GCA TCC CAA AG3′ |

| | | | |
|---|---|---|---|
| Alleles | Length (bp) | (CA)n | Frequency |

| | | | |
|---|---|---|---|
| 1 | 205 | 24 | 0.8807 |
| 2 | 203 | 23 | 0.0148 |
| 3 | 201 | 22 | 0.0875 |
| 4 | 119 | 21 | 0.0000 |
| 5 | 117 | 19 | 0.0000 |
| 6 | 115 | 18 | 0.0136 |
| 7 | 113 | 17 | 0.0011 |
| 8 | 111 | 16 | 0.0000 |
| 9 | 99 | 15 | 0.0000 |
| 10 | 97 | 14 | 0.0023 |

| | |
|---|---|
| Heterozygosity: | 0.2163 |
| No. of chromosomes: | 880 |

| | |
|---|---|
| D8S2614 | |
| Primers: | 5′GAC CCA CTG CCA CAC TCT TT3′ |
| | 5′GGA GTG CGG CAT GAA ATT AT3′ |

| | | | |
|---|---|---|---|
| Alleles | Length (bp) | (CA)n | Frequency |

| | | | |
|---|---|---|---|
| 1 | 194 | 22 | 0.0057 |
| 2 | 192 | 21 | 0.0588 |
| 3 | 190 | 20 | 0.4570 |
| 4 | 188 | 19 | 0.0916 |
| 5 | 186 | 18 | 0.1437 |
| 6 | 184 | 17 | 0.0045 |
| 7 | 182 | 16 | 0.2285 |
| 8 | 180 | 15 | 0.0000 |
| 9 | 178 | 14 | 0.0011 |
| 10 | 176 | 13 | 0.0079 |
| 11 | 174 | 12 | 0.0011 |

| | |
|---|---|
| Heterozygosity: | 0.7063 |
| No. of chromosomes: | 884 |

| | |
|---|---|
| D8S2613 | |
| Primers: | 5′ATA TGT ATA CAA TGT GTA TCT GTA TC3′ |
| | 5′CCT TTT AGT TCC CAT TCC CAT T3′ |

| | | | |
|---|---|---|---|
| Alleles | Length (bp) | (CA)n | Frequency |

| | | | |
|---|---|---|---|
| 1 | 133 | 16 | 0.0275 |
| 2 | 131 | 15 | 0.2002 |
| 3 | 129 | 14 | 0.1167 |
| 4 | 127 | 13 | 0.0000 |
| 5 | 125 | 12 | 0.6362 |
| 6 | 123 | 11 | 0.0000 |
| 7 | 121 | 10 | 0.0046 |
| 8 | 119 | 9 | 0.0000 |
| 9 | 117 | 8 | 0.0137 |
| 10 | 115 | 7 | 0.0011 |

| | |
|---|---|
| Heterozygosity: | 0.5406 |
| No. of chromosomes: | 874 |

| | |
|---|---|
| D8S2618 | |
| Primers: | 5′TGA TGC AGG AGA ATT GCT TG3′ |
| | 5′CCT ACT TGG CTG GGA TTC TG3′ |

| | | | |
|---|---|---|---|
| Alleles | Length (bp) | (CA)n | Frequency |

| | | | |
|---|---|---|---|
| 1 | 182 | 24 | 0.00457 |
| 2 | 180 | 23 | 0.00686 |
| 3 | 178 | 22 | 0.05148 |
| 4 | 176 | 21 | 0.01487 |
| 5 | 174 | 20 | 0.07208 |
| 6 | 172 | 19 | 0.04462 |
| 7 | 170 | 18 | 0.02745 |
| 8 | 168 | 17 | 0.36041 |
| 9 | 166 | 16 | 0.02288 |
| 10 | 164 | 15 | 0.00228 |
| 11 | 162 | 14 | 0.01029 |
| 12 | 160 | 13 | 0.37185 |
| 13 | 158 | 12 | 0.00000 |
| 14 | 156 | 11 | 0.00000 |
| 15 | 154 | 10 | 0.00915 |
| 16 | 152 | 9 | 0.00114 |

| | |
|---|---|
| Heterozygosity: | 0.72023 |
| No. of chromosomes: | 874 |

Three neighbouring polymorphisms, D8S66181, D8S6687 and D8S261, spanning a region of approximately 108 kb demonstrated significant evidence for allelic association with schizophrenia with a p-value 0.004, 0.024 and 0.055 respectively after a Monte Carlo correction for multiple alleles (Table 2). Statistically significant marker-to-marker linkage disequilibrium was found between the positively associated markers in this region and overall correlated well with the physical distance of the markers. The new microsatellite and SNP markers identified via sequencing and genotyping were genotyped in an LD analysis of samples collected from cases of schizophrenia. The results of this LD analysis narrowed down the chromosome 8p arm region within which a gene involved in neuropsychiatric disorders lies to an interval of 70 kb flanking the publicly available marker D8S261. The gene PCM1 was the only gene mapping exactly at the positions of maximum disequilibrium over the 70 kb region thus showing it can be involved in neuropsychiatric disorders.

TABLE 3
CLUMP test results for association between schizophrenia
and alleles at novel polymorphic loci on chromosome 8p21.3-22 in a
UK case-control sample

| Marker | T1 | | T2 | | T3 | | T4 | |
|---|---|---|---|---|---|---|---|---|
| Locus | X² | p-value | X² | p-value | X² | p-value | X² | p-value |
| (tel→cen) | 8.191 | 0.215 | 3.439 | 0.497 | 3.271 | 0.287 | 3.795 | 0.414 |
| D8S2612 | | | | | | | | |
| D8S2617 | 6.714 | 0.465 | 4.139 | 0.510 | 1.965 | 0.570 | 1.965 | 0.797 |
| AFM333th1 | 5.301 | 0.509 | 4.108 | 0.544 | 2.061 | 0.512 | 3.744 | 0.426 |
| D8S261 | 16.67 | 0.055 | 10.60 | 0.150 | 9.992 | 0.010 | 13.82 | 0.017 |
| D8S2616 | 19.92 | 0.024 | 12.99 | 0.048 | 5.268 | 0.118 | 11.91 | 0.031 |
| D8S2615 | 15.17 | 0.004 | 10.54 | 0.009 | 10.54 | 0.005 | 12.12 | 0.004 |
| D8S2614 | 6.560 | 0.720 | 4.882 | 0.445 | 3.551 | 0.260 | 4.164 | 0.474 |
| D8S2613 | 5.449 | 0.496 | 2.728 | 0.608 | 0.273 | 0.956 | 3.937 | 0.319 |
| D8S2618 | 12.36 | 0.521 | 7.996 | 0.423 | 0.790 | 0.947 | 9.345 | 0.210 |

Where,
T1: a straightforward Pearson's χ² statistic of the ‘raw’ contingency table
T2: the χ² statistic of a table with rare alleles grouped together to prevent small expected cell counts
T3: the largest of the χ² statistics of 2 × 2 tables each of which compares one allele against the rest grouped together
T4: the largest of the χ² statistics of all possible 2 × 2 tables comparing any combination of alleles against the rest

The pericentriolar material 1 gene (PCM 1) has been mapped to chromosome 8p21.3-33 [Ohata, (1994)Genomics 24, 404-406)

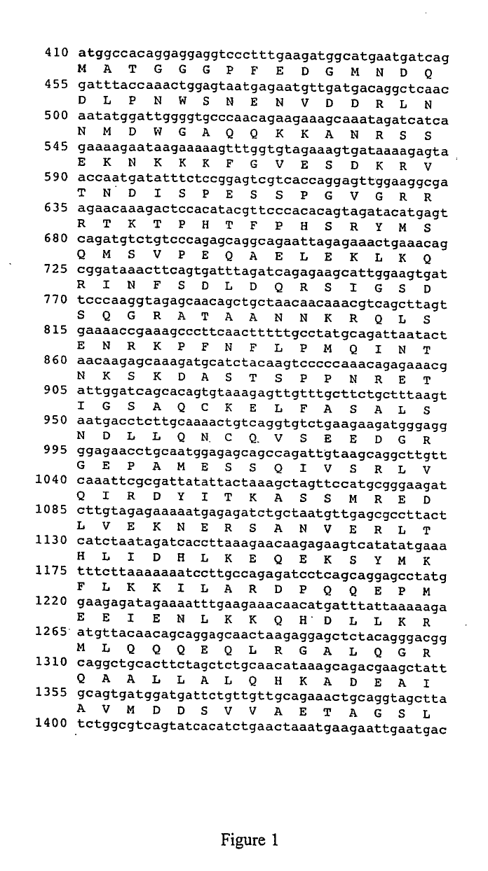

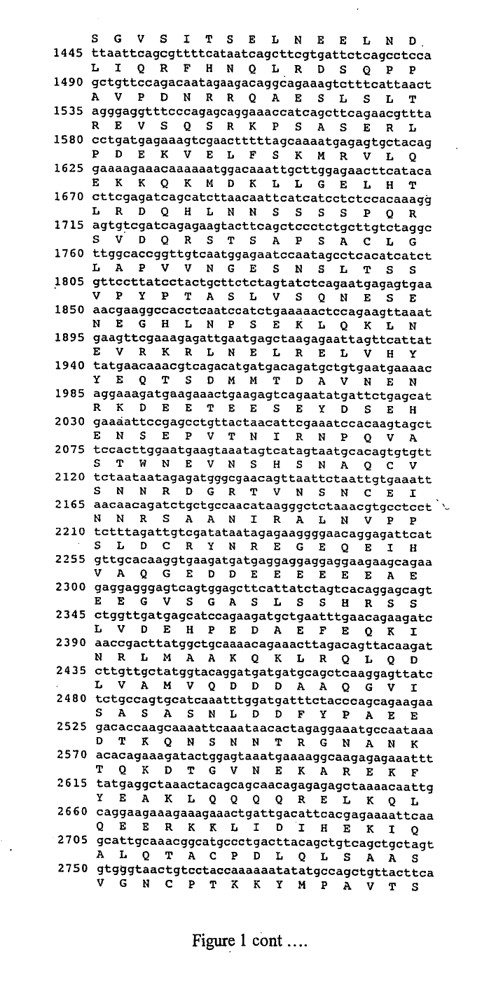

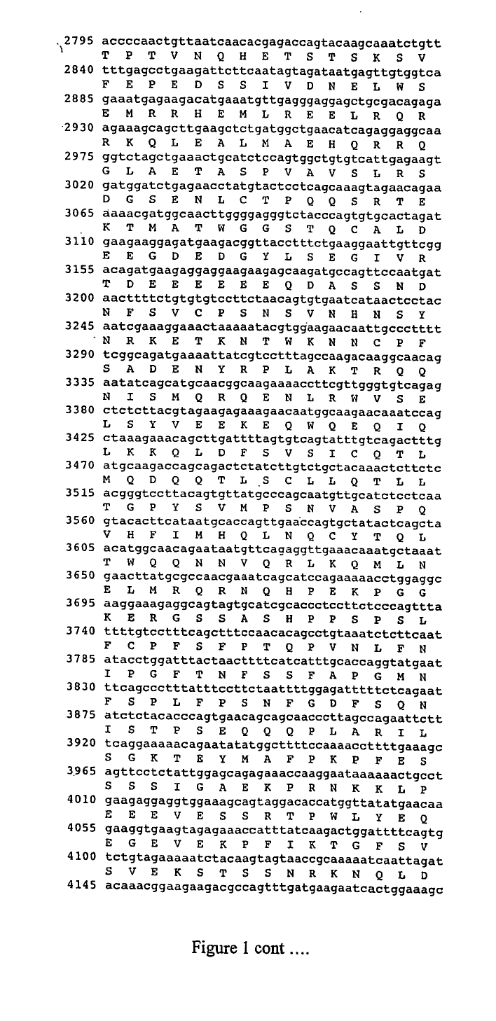

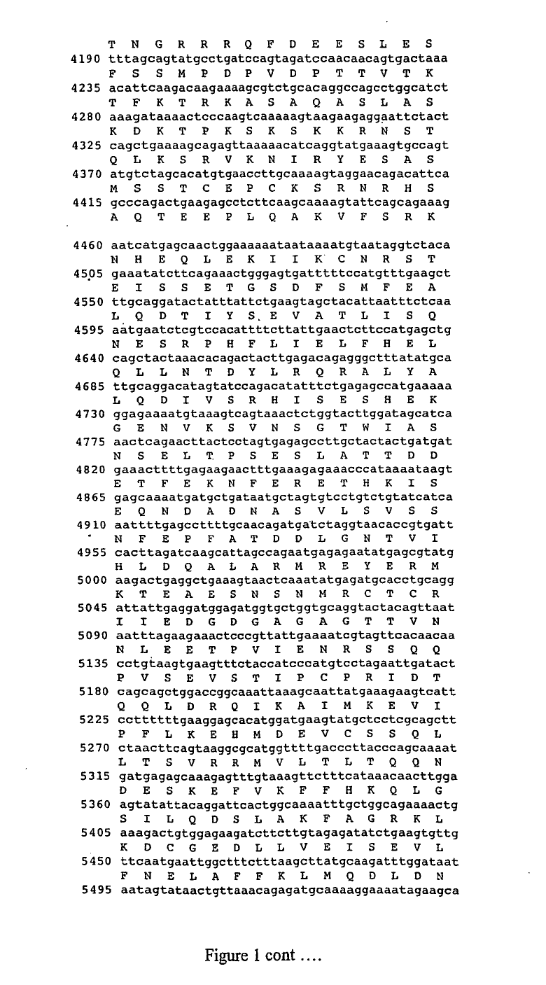

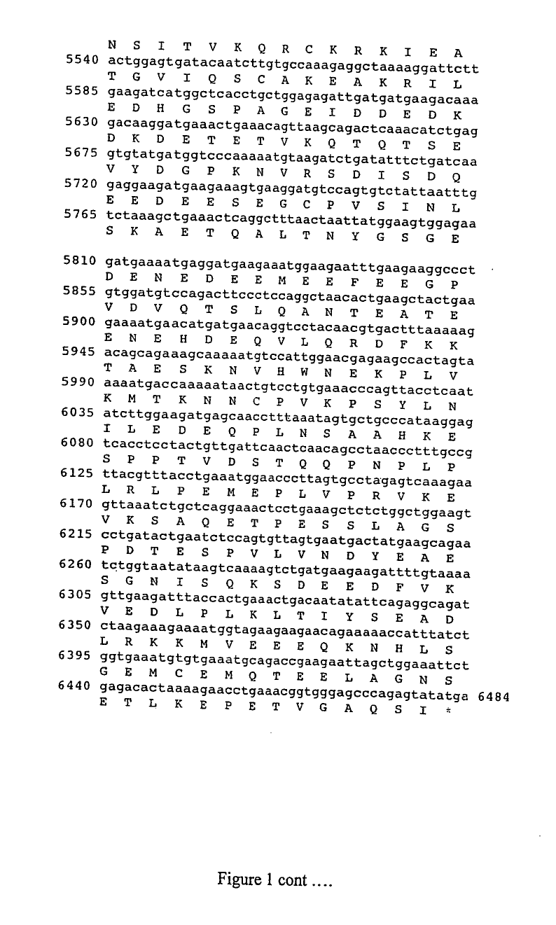

In addition, it is annotated in the Genome Channel and its sequence occupies part of clones AB020866 and AB020867 of the NT_{—}000501 contig. Two of the associated polymorphisms, D8S2616 and D8S261 lie within the intronic sequence of this gene while the third polymorphism, D8S2615 resides approximately 75 kb upstream of the translation initiation codon of this gene. Balczon et al, [Balczon, 1991, Cell Motil Cytoskeleton 20:121-135] identified a ˜220 kD centrosome autoantigen. A cDNA encoding the entire protein was isolated from a human fetal liver cDNA library. Analysis of the cloned sequence identified an open reading frame of 6,072 nucleotides encoding 2,024 amino acids. From the deduced amino acid sequence, the exact molecular mass of PCM1 was calculated to be 228,705 daltons [Balczon, 1994]. PCM1 nucleotide and amino acid sequences are shown in FIG. 1.

### EXAMPLE 2

### Genomic Organization of PCM1

The predicted coding sequence has a translation initiation codon (ATG) located at position 410 of PCM1 mRNA, which is preceded by a Kozak consensus (CCAXXATGG) initiation sequence [Kozak, 1984, Nature 308: 241-246] and a stop codon (TGA) is located at position 6482 of the PCM1 mRNA (FIG. 1).

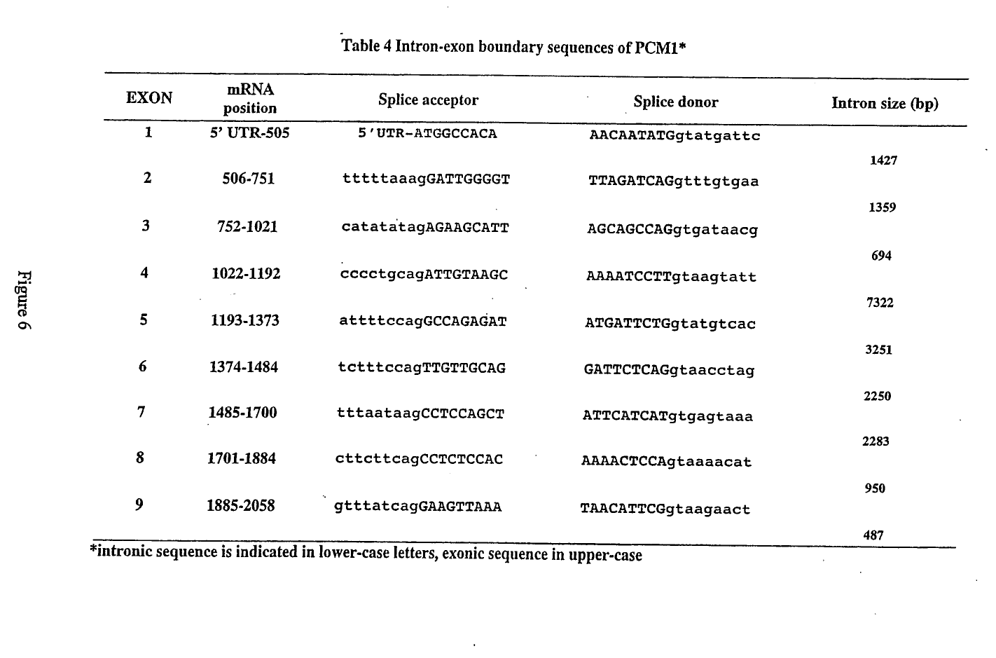

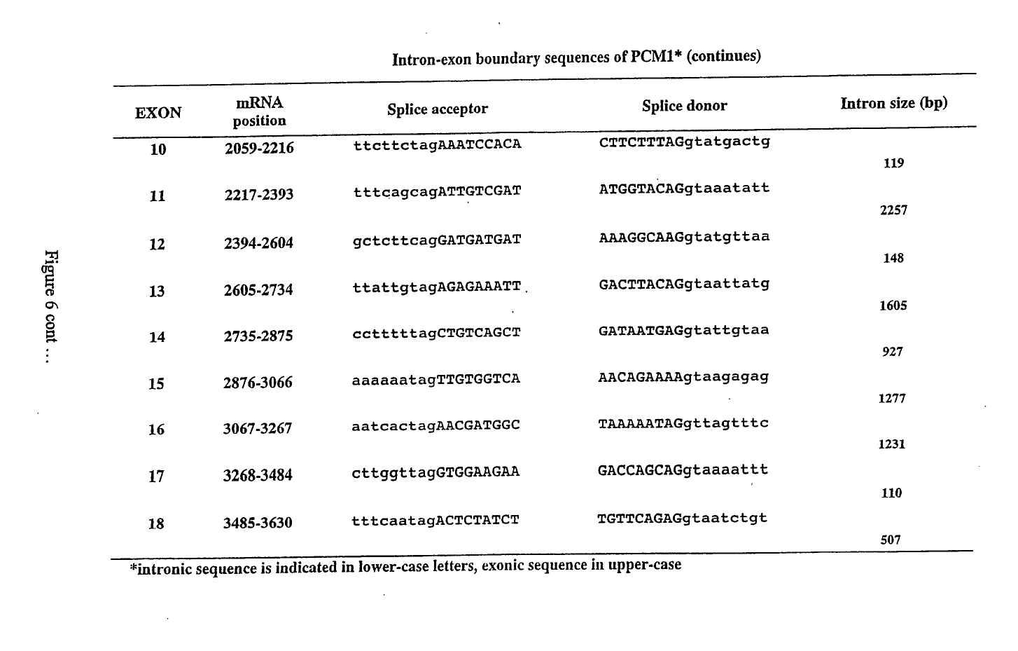

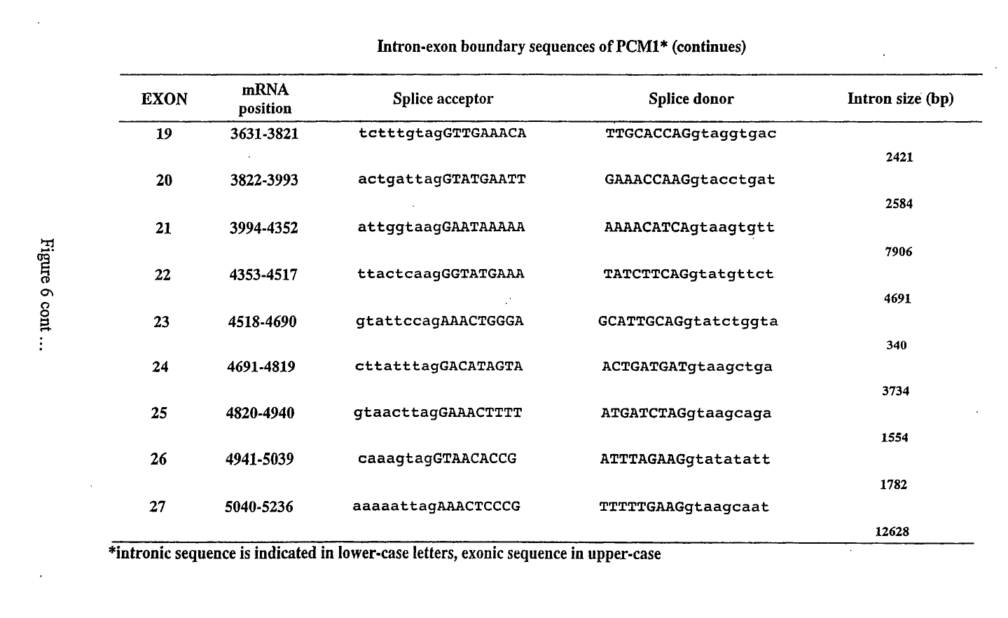

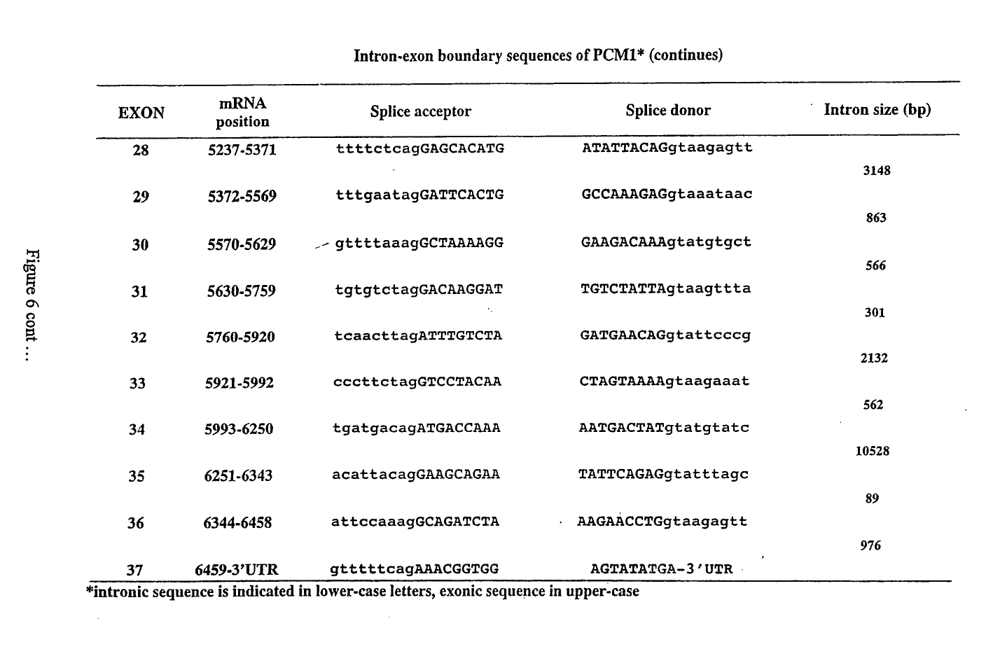

In order to define the exon-intron genomic organization, “BLAST 2 sequences” program from the NCBI was used. This is a tool, which produces an alignment of two given sequences using the BLAST program. In this case, the two sequences were the mRNA sequence of PCM1 (Accession No. NM_{—}006197) against the corresponding genomic sequence (clones AB020866 and AB020867 of the NT_{—}000501 contig) The exon-intron junctions of the PCM1 transcript derived from these data is shown in Table 4 (FIG. 6). The predicted exon-intron boundaries are in excellent agreement with the “GT-AG rule” that is nucleotides at the exon-intron boundaries are not random but introns always seem to begin with GT and end with AG. In addition, the predicted exons are in agreement with the exons generated by the GRAILEXP program used for gene prediction by the Genome Channel, which is a site that provides automated annotation of genomic sequence.

The coding sequence has a length of 6075 nt and the translated product is 2024 aa long. The translation initiation codon (atg) is at position 410 while the stop codon (tga) is at position 6482 of the PCM1 mRNA. Obtained form the National Institute of Biotechnology Information (NCBI) Open Reading Frame finder (http://www.ncbi.nlm.nih.gov/gorf/gorf.html)

The data indicate that the PCM1 gene is composed of 37 exons spread over an area of approximately 92 kb of genomic sequence and cover perfectly the whole 6075 nt of the PCM1 open reading frame.

Furthermore, Nucleotide Identify X (NIX, http://menu.hgmp.mrc.ac.uk/menu-bin/Nix/Nix.pl) analysis of 20 kb of genomic sequence upstream of the translation initiation codon identified a 868 nt putative CpG island (67% GC) encompassing part of the 5′ end of the PCM1 transcript. Blast 2.0 homology search revealed perfect match of part of this CpG island with a CpG island isolated by the Sanger Centre [Cross, 1994, Nature Genetics 6, 236-244). Potential promoter sites were also predicted around this CpG island. A potential consensus polyadenylation signal is present 63 nt downstream of the stop codon.

Exon 37
gtttttcagAAACGGTGGGAGCCCAGAGTATATGAGATGTCTTCAGAGGCTCATCTAACTCTGTCCT
TACATACTCAATGCATATATGAAAACAATACTAAATAAACATCTGATCTGTATAAAAATGTAA

PCM1 is represented by several ESTs in the Unigene database (http://www.ncbi.nlm.nih.gov/UniGene) and has been assigned the Unigene identification number Hs.75737. Expression data available in Unigene show that PCM1 is expressed in a wide variety of tissues (adrenal gland, aorta, bone, brain, breast, CNS, colon, eye, germ cell, head and neck, heart, kidney, lung, lymph, muscle, ovary, pancreas, parathyroid, placenta, prostate, smooth muscle, spleen, stomach, testis, thyroid, tonsil, uterus, whole embryo), including brain.

The open reading frame of PCM1 encodes a putative protein of 2024 amino acids. A consensus nucleotide-binding site extending from amino acids 1167-1174 was found. This amino acid stretch of ARILSGKT corresponds to the consenus ATP/GTP binding motif that has been identified in several ATPases, kinases and GTP-binding proteins. In addition, it has the potential of forming coiled-coils.

### EXAMPLE 3

### Analysis of PCM1 DNA Variation by Automated Bi-directional DNA Sequencing of Amplified Exons

For this analysis, 19 schizophrenia cases from the sample used in the association study together with 2 cases from each British multiply affected schizophrenia family that showed positive lods on chromosome 8p21-22 (26 affected individuals) and 10 unrelated healthy controls were used. In order to increase the chances of detecting a mutation in linkage disequilibrium with schizophrenia, affected individuals that carried the alleles with higher frequencies in the cases than in the controls in the allelic association study that was described in the previous chapter for the microsatellite markers D8S261, D8S2616 and D8S2615 were chosen.

PCM1 consists of 37 exons spanning about 92 kb of genomic sequence. Each exon is however relatively small ranging in size from about 60 to 360 bp. Marker D8S2616 lies within the intronic sequence of PCM1 between exons 4 and 5, while D8S261 is between exons 19 and 20.

The strategy chosen for mutation and DNA variation screening was automated bi-directional sequencing of PCR amplified exons. The whole exonic sequence as well as more than 100 bp of intronic sequence on either side of each exon were PCR amplified using M13 tailed primers as described. The PCR product was then sequenced using the Thermo Sequenase kit. Each sequencing reaction contains two different M13 primers labelled with different IR dyes (IRD700 and IRD800) in order to obtain sequence from both DNA strands of the PCR product (bi-directional sequencing). In this way, both strands of the PCR products can be completely sequenced. This approach is particularly useful for mutation detection as both forward and reverse strands are assessed simultaneously for the presence of a variation. In addition, detection of heterozygotes is highly accurate. Furthermore, this sequence-based method can detect the specific location of each mutation. Finally, the use of the same sequencing primers for all PCR products makes bi-directional tailed primer sequencing economically feasible. Therefore, direct sequencing on the LI-COR was preferred to a combination of SSCP and sequencing as a more reliable and faster approach.

LI-COR's dual-dye automated DNA sequencer (Model 4200 IR² System) was used to electrophorese and analyse the forward and reverse sequences of the bi-directional sequencing reactions in parallel. Sequence data were interpreted using the automated base-calling algorithms of LI-COR's Base ImagIR™ software.

Automatic alignment was performed by LI-COR's AlignIR™ software. The software aligns the sequences for the forward and reverse strand from all the samples and produces a consensus. The consensus was generated by a minimum method that considers all base letters and generates an IUPAC ambiguity code for combination of bases at that position. The consensus sequence is shown in a row below the last sample sequence while an annotation row below makes it easy to see ambiguities and mismatches in the consensus sequence. When an ambiguity or a mismatch was encountered at a certain position, windows containing the chromatogram files (.scf files) for both forward and reverse strands for all the samples could be opened in order to assess the validity of the base calls. Wrong base calls were edited in the AlignIR window by just highlighting the sequence and typing the appropriate letter or the IUPAC ambiguity code.

Heterozygous sites were identified by scanning the assembled sequence traces for: (i) the presence of a drop in fluorescence peak height at a position when compared to the respective peak height for all individuals that are homozygous at the position and (ii) presence of another base (a second peak) that accompanies the drop in fluorescence peak height.

Further polymorphisms and variants have also been identified as follows:
A to G SNP at base number 75915 of clone AB020866
T to C SNP at base number 79215 of clone AB020866
G to A SNP at base number 80123 of clone AB020866
C to T SNP at base number 87366 of clone AB020866
G to A SNP at base number 96961 of clone AB020866
G to A SNP at base number 97759 of clone AB020866
A to C SNP at base number 510 of clone AB020867
C to T SNP at base number 2450 of clone AB020867
Insertion/Deletion CC to C at base number 10153 of clone AB020867
A to G SNP at base number 30364 of clone AB020867
T to C SNP at base number 32005 of clone AB020867
C to T SNP at base number 61847 of clone AB020867
A to C SNP at base number 51659 of clone AB020867
T to C SNP at base number 78722 of clone AB020867
T to C SNP at base number 75925 of clone AB020866
A to G SNP at base number 79214 of clone AB020866
A to G SNP at base number 80254 of clone AB020866
G to A SNP at base number 87507 of clone AB020866
A to C SNP at base number 61873 of clone AB020867
In/Del at base number 51745 of clone AB020867

It will be understood that the invention encompasses also these markers and those in linkage disequilibrium with them.

### EXAMPLE 4

### Analysis of Exon 4 and its Exon/Intron Boundaries

Exon 4 was screened first for mutations and DNA variation because marker D8S2616 that showed evidence of allelic association with schizophrenia lies within the intronic sequence of PCM1 between exons 4 and 5.

A sequence of 507 bp was PCR amplified using M13 tailed primers. The sequence of the primers has as follows:

```
5′-GGATAACAATTTCACACAGG-CCAAGTGTCTTTGGTTATCTTCG-3′
      M13 reverse primer (−21) forward PCR primer
5′-CACGACGTTGTAAAACGAC-AGTCCGAACATCCTCCTCCT-3′
      M13 forward primer (−29) reverse PCR primer
```

This product included the whole of exon 4 (170 bp) while the rest of the amplified sequence was intronic sequence from both sides of this exon. PCR products were subsequently sequenced bi-directionally. Sequence was obtained for all 10 controls and 45 cases.

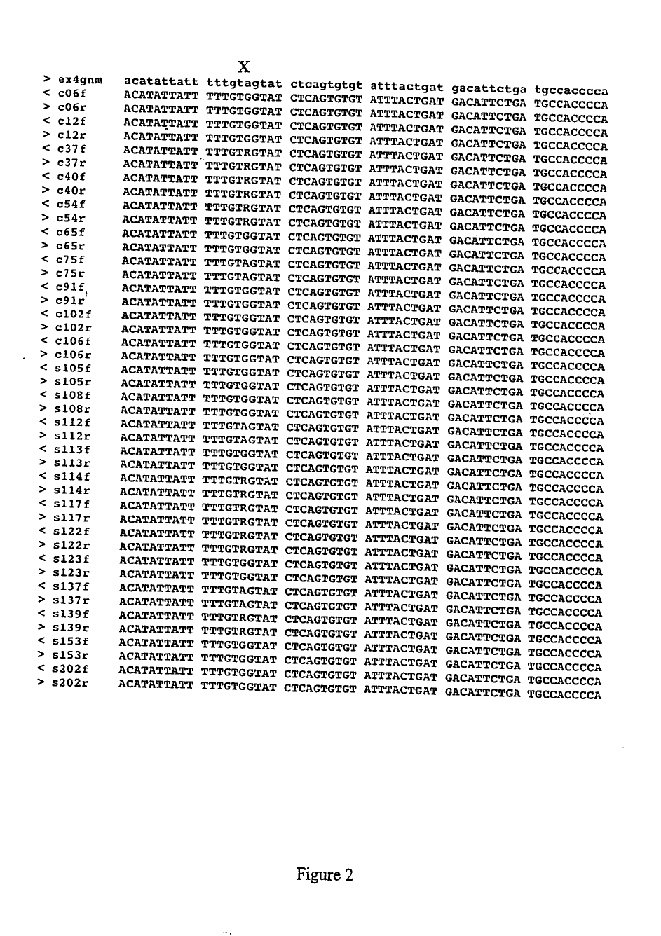

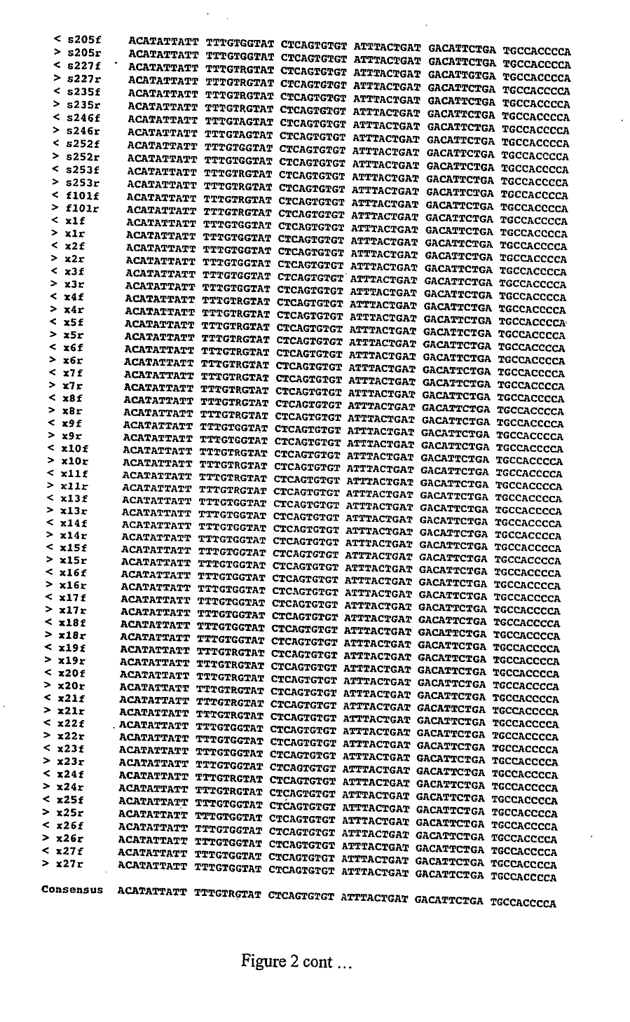

Sequence analysis by LI-COR's AlignIR™ software followed. A single nucleotide polymorphism was identified in the intronic sequence that was 3′ to the exon. This is an A to G substitution that occurred in both patients and controls. Overall, 6 GG homozygotes, 1 AA homozygote and 3 heterozygotes were identified in the samples from the control individuals while 23 GG homozygotes, 3 AA, homozygotes and 19 heterozygotes were identified in the individuals with schizophrenia. The DNA variation occurs at nucleotide position 80254 in the AB020866 genomic clone. The AlignIR report that contains the novel DNA variant identified in PCM1 using sequence analysis is presented in FIG. 2.

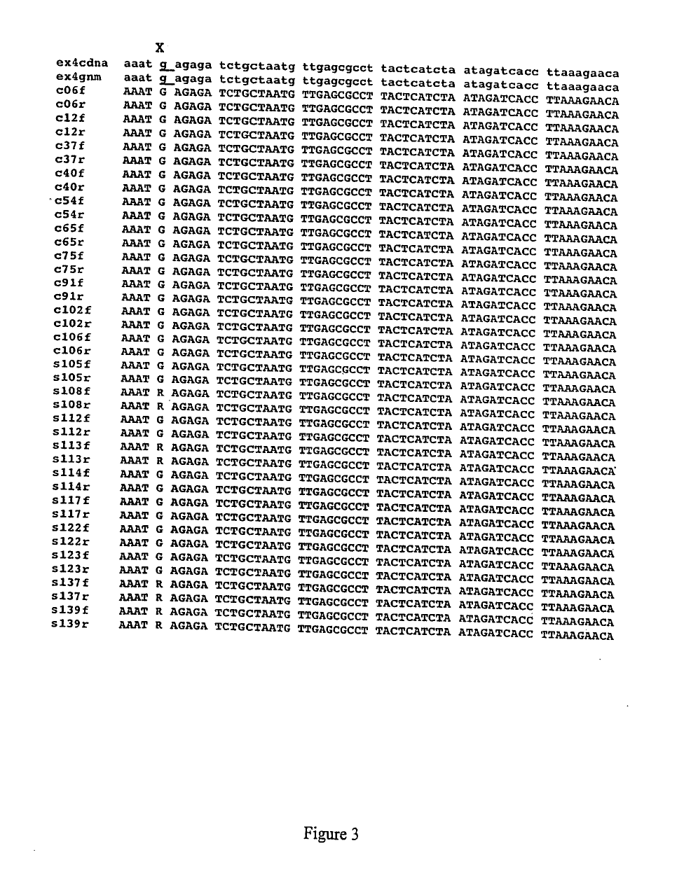

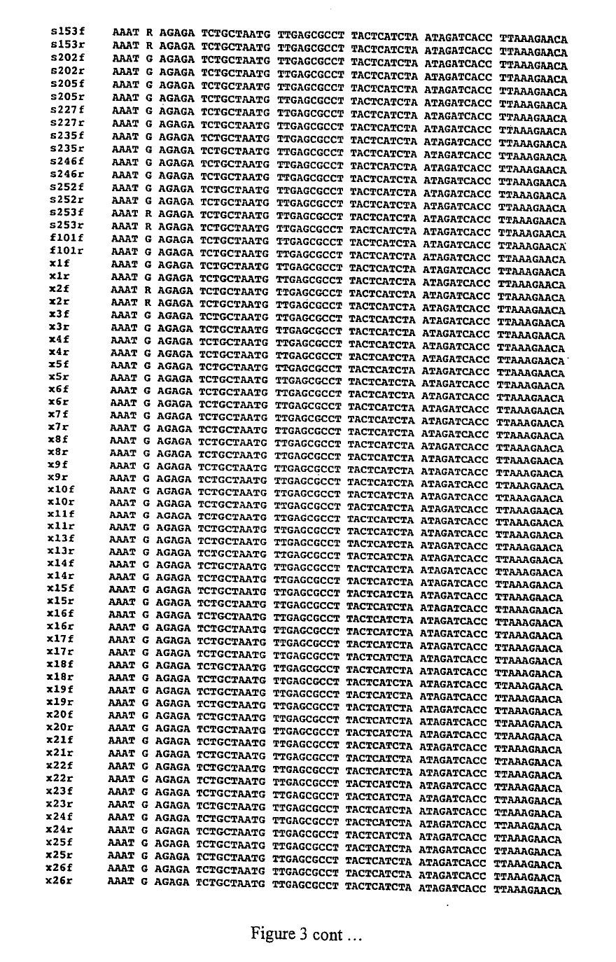

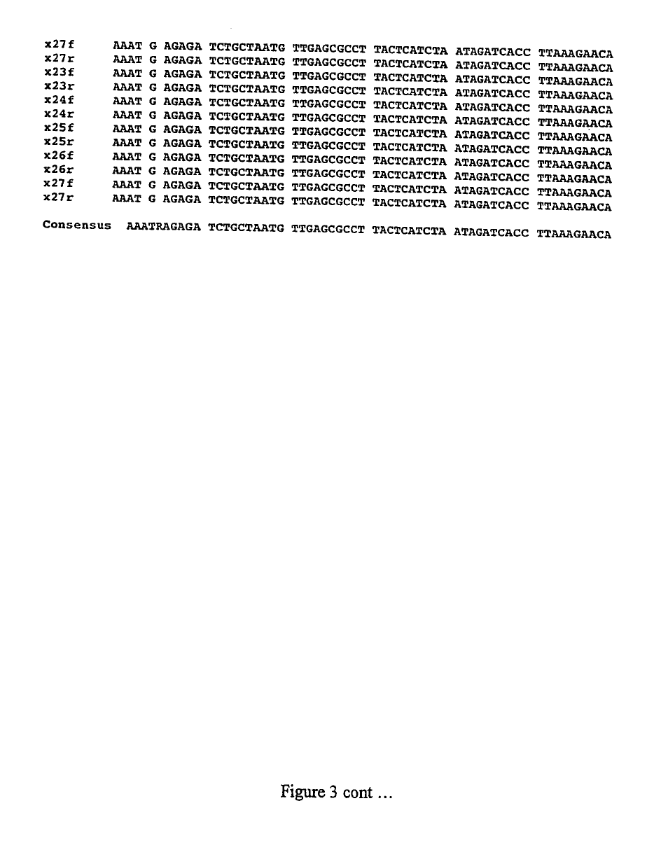

A candidate single nucleotide polymorphism was identified in the coding sequence at position 1100 of the PCM1 mRNA (NM_{—}006197) and 80123 of the AB020866 clone. It is a G to A substitution with relatively low frequency as only GG homozygotes and a few heterozygotes were observed. More specifically, 7 heterozygotes were observed among the cases while there were no heterozygotes in the controls. The rest of the individuals were GG homozygotes. This is a very interesting polymorphisms as it seems to produce an amino acid change in the protein sequence from alanine (gct) to threonine (act). More individuals will have to be examined in order to validate this polymorphism. The AlignIR report that contains this DNA variant is presented on FIG. 3.

The sequence of clone AB020866 was the reference genomic sequence obtained from the Entrez nucleotide database and can be seen at the first line (ex4gnm) of FIG. 2. A variation occurs at nucleotide position 80254 of the AB020866 clone. The consensus sequence obtained from AlignIR can be seen at the last line (Consensus). Heterozygotes, are marked R, G homozygotes are marked G and A homozygotes are marked A]

### EXAMPLE 5

### Analysis of Exon 5 and its Exon/Intron Boundaries

Exon 5 was screened for mutations and DNA variation at this stage because marker D8S2616 that showed evidence of allelic association with schizophrenia lies within the intronic sequence of PCM1 between exons 4 and 5.

A sequence of 595 bp was PCR amplified using M13 tailed primers. The sequence of the primers has as follows:

```
5′-GGATAACAATTTCACACAGG-TGAGCCATTGATTATG-3′
          M13 reverse primer (−21) forward PCR primer
5′-CACGACGTTGTAAAACGAC-AGTTGTCCCTGCAACCT-3′
          M13 forward primer (−29) reverse PCR primer
```

This product included the whole of exon 5 (180 bp) while the rest of the amplified sequence was intronic sequence from both sides of this exon. PCR products were subsequently sequenced bi-directionally. Sequence was obtained for 5 controls and 42 cases.

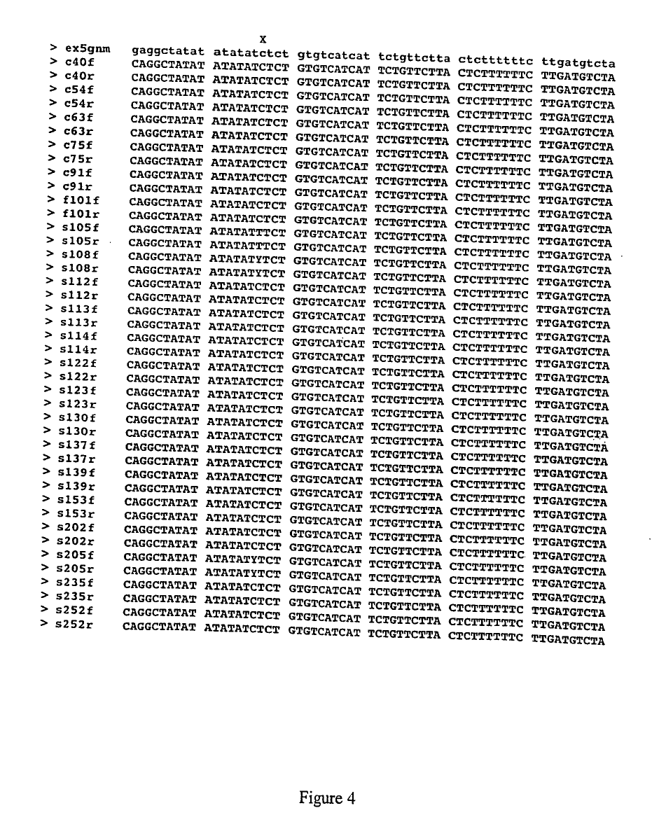

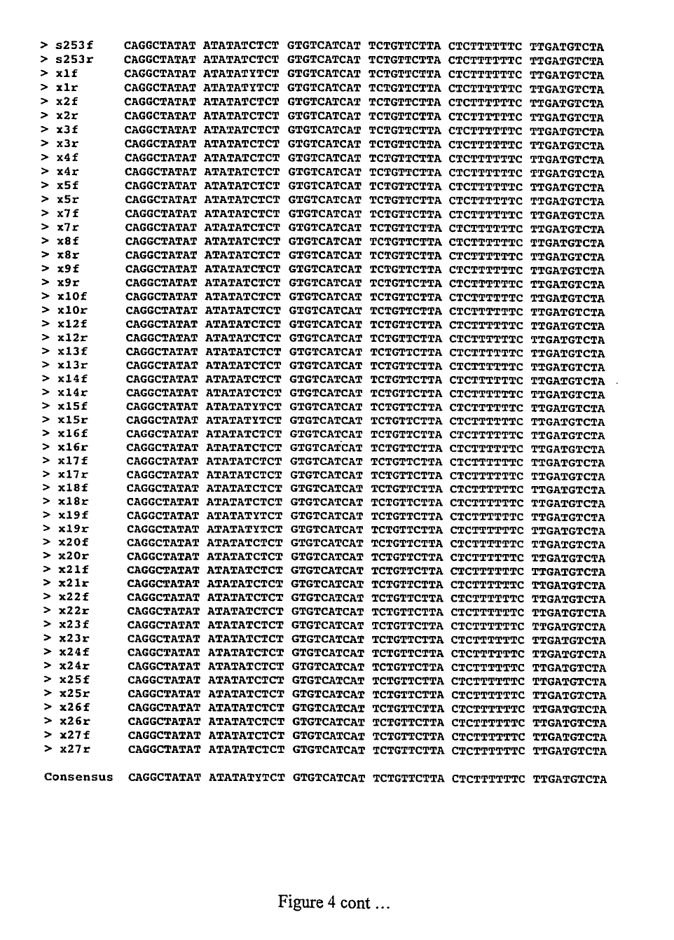

Sequence analysis by LI-COR's AlignIR™ software followed. Two single nucleotide polymorphisms were identified in the intronic sequence that was 5′ to exon 5. One is a C to T substitution occurring 171 bp from the start of exon 5 at position 87366 of the AB020866 genomic clone. In the individuals examined, 1 TT homozygote and 5 heterozygotes were identified among the cases while the rest were CC homozygotes including all 5 controls. The AlignIR report that contains this DNA variant is presented on FIG. 4.

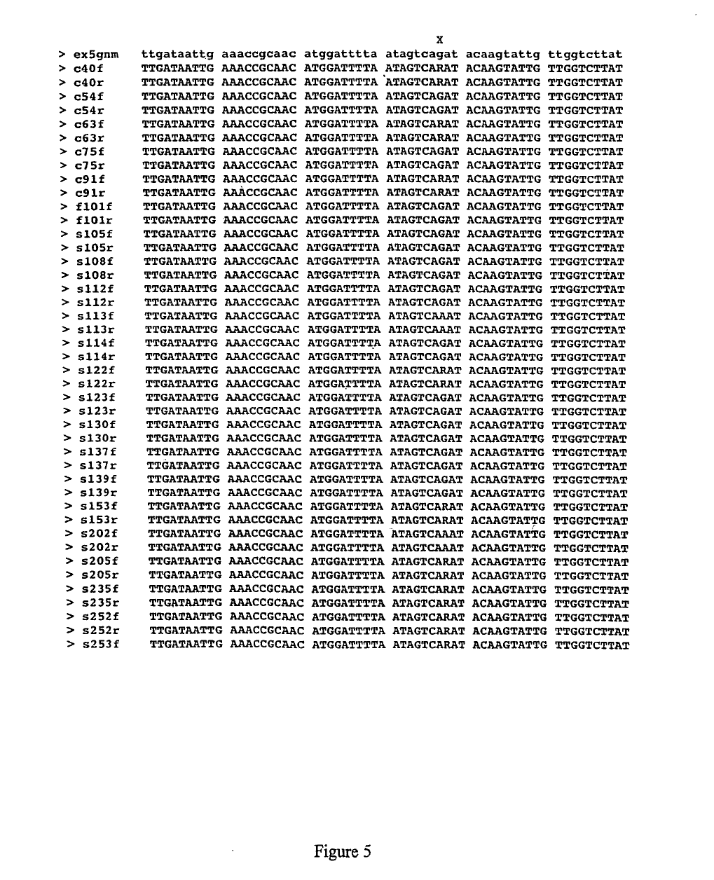

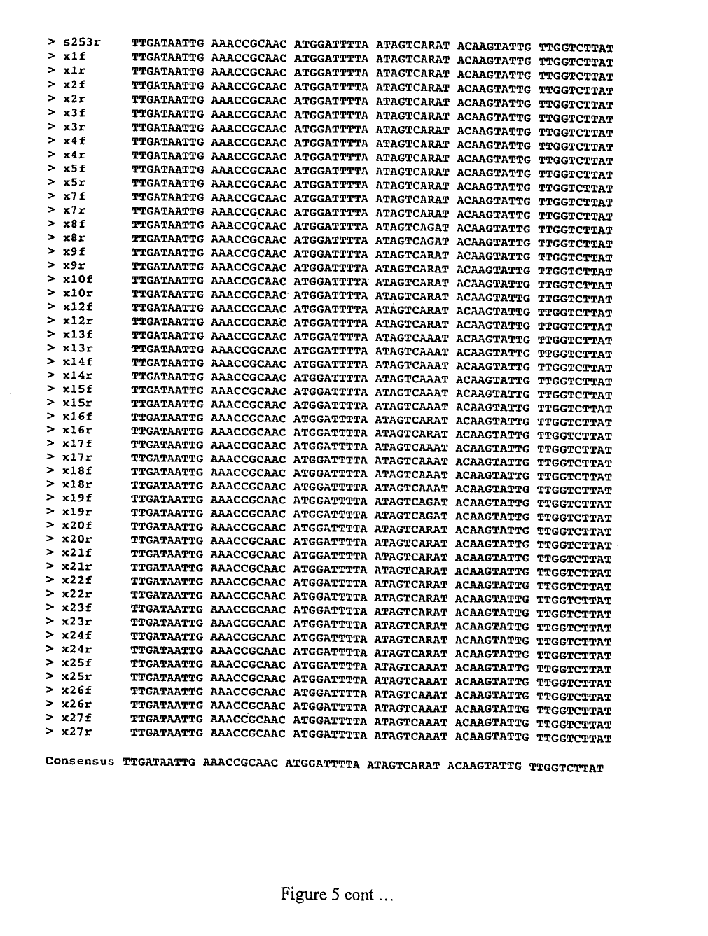

A second single nucleotide polymorphism which is a G to A substitution occurring 30 bp 5′ to the start of the exon at nucleotide position 87507 of the AB020866 genomic clone. 2 GG homozygotes and 3 heterozygotes were identified in the controls examined while 11 GG homozygotes, 10 AA homozygotes and 21 heterozygotes were identified in the cases. The AlignIR report that contains this DNA variant is presented on FIG. 5.

All 4 single nucleotide polymorphisms identified in and around exons 4 and 5 of the PCM1 were examined for possible alteration in a restriction enzyme cutting site but none seemed to produce or obliterate one.

Discussion

PCM1 is part of the centrosome assembly. Although, possible roles in the maintenance of centrosome integrity and the regulation of the microtubule cytoskeleton have been mentioned its function in neuropsychiatric disorders has not previously been elucidated. Its protein structure bears similarities to myosins, structural proteins and proteins involved in motility and/or transport (microtubule binding proteins). As is described by Millar et al. [2000] many such structural proteins are implicated in processes such as axon guidance, synaptogenesis, functioning of the synapse and intracellular transport along axons and dendrites. It is now the case that PCM1 has a similar role implicating this protein in the development of the nervous system and/or neuronal activity and is therefore involved in the aetiology of schizophrenia, PCM1 disorders and neuropsychiatric disorders.

Furthermore, PCM1 interacts with a brain-specific protein, huntingtin-associated protein 1 (HAP1). HAP1 binds to huntingtin in a glutamine repeat length-dependent manner as well as interacts with cytoskeletal, vesicular and motor proteins. In this way, it acts as an adaptor protein in order to mediate interactions among all these different molecules [Engelender, 1997, Hum Mol Genet 6: 2205-2212].

The fact that PCM1 interacts with proteins that are directly involved with neuronal function indicates that it may be involved in wide network of proteins responsible for the normal functioning of neuronal cells.

In order to identify mutations as well as DNA sequence variation the method of fluorescence bi-directional DNA sequencing was employed. DNA sequencing is the most sensitive method for finding DNA polymorphisms and mutations. In addition to its sensitivity, full DNA sequence analysis provides complete knowledge of the type, position and context of every variation, regardless of whether it is a single nucleotide substitution or an insertion/deletion variation.

So far, two of the exons, exon 4 and exon 5 together with at least 100 bp of intronic sequence on either side of each exon have been screened for single nucleotide polymorphisms. These exons were screened first because marker D8S2616 that showed evidence for allelic association with schizophrenia lies in the intronic sequence between these two exons.

In exon 4, a G to A transition was identified at the 3′ intronic sequence of this exon in both patients and controls. It seems to be a frequent variant as homozygotes for each of the alternative alleles are present in the individuals screened as well as a number of heterozygotes containing these alternative alleles. In addition, a single nucleotide polymorphisms was identified in exon 4. It is again a G to A transition but the relative frequency of this variant is low because it was identified by infrequent heterozygotes among homozygotes. This polymorphism was identified as heterozygotes amongst individuals identified as being schizophrenic and was not found in normal controls. It produces an amino acid change, from an alanine to a threonine, in the predicted protein sequence of pericentriolar material 1. Such a muations has been found to be associated with diseases in at least 140 other instances.

Two single nucleotide polymorphisms were identified in the intronic sequence 5′ to exon 5. Both substitutions are transitions, one from a C to a T and the other from a G to an A and are 140 bp apart.

Although only a small part of the genomic sequence of PCM1 has been screened so far, the results are consistent with other studies of human DNA sequence variation, which have identified on average 1 variable site every 217 bp [Halushka, 1999, Nat Genet 22: 239-247]. Furthermore, all the nucleotide substitutions identified in this study were transitions, which is in agreement with other studies [Halushka, 1999, ref as above]. Especially, A/G substitutions have the highest prevalence among functional genes [Gojobori, 1982, J Mol Evol 18, 414-423]. Further screening of the PCM1 regulatory and coding sequence are likely to yield other types of mutations causing schizophrenia and other neuropsychiatric disorders and PCM1 disorders. In the light of the present disclosure anyone skilled in the art of sequencing will find such mutations in increasing numbers of patients.

1. A method for determining the susceptibility of an individual to a neuropsychiatric disorder, or a method of diagnosis or prognosis of the neuropsychiatric disorder, the method comprising use of a marker located in the chromosomal region 8p21-22.

2. A method as claimed in claim 1 wherein the disorder is schizophrenia.

3. A method as claimed in claim 1 or claim 2 wherein the method comprises:
(i) obtaining a nucleic acid or protein sample from the individual;
(ii) determining the structure, level of expression, and\or activity of the polypeptide encoded by the pericentriolar material 1 (PCM1) gene.

4. A method as claimed in claim 1 or claim 2 wherein the method comprises:
(i)obtaining a sample of nucleic acid from the individual, and
(ii)determining in that sample, the presence or absence of a pericentriolar material 1 (PCM 1) marker.

5. A method as claimed in claim 4 wherein the nucleic acid is RNA, cDNA or genomic DNA.

6. A method as claimed in claim 4 or claim 5 wherein the PCM1 marker is selected from the group consisting of the any of the following microsatellite repeats present in the NT_{—}000501 contig on chromosome 8p21.3-22: D8S2612; D8S2613; D8S2614; D8S2615; D8S2616; D8S2617; D8S2618, or D8S261, or a polymorphic marker which is in linkage disequilibrium with any of these.

7. A method as claimed in claim 5 wherein the PCM1 marker is: D8S261; D8S2615; or D8S2616;

8. A method as claimed in any one of claims 4 to 7 wherein the PCM1 marker is within the PCM1 gene.

9. A method as claimed in claim 8 wherein the PCM1 marker is an in the intronic sequence 3′ to exon 4; in exon 4; in the intronic sequence 5′ of exon 5 wherein said exon-intron are set out in FIG. 6.

10. A method as claimed in claim 9 wherein the PCM1 marker is an SNP selected from the group consisting of the following positions numbered in accordance with the sequence of clone AB020866 as set out FIGS. 2-5:
(i) 80254,
(ii) 80123
(iii) 87366
(iv) 87507
or a polymorphic marker which is in linkage disequilibrium with any of these.

11. A method as claimed in claim 10 wherein the identity of the nucleotide at the SNP is shown at the corresponding numbered position in any one of FIGS. 2-5.

12. A method as claimed in claims 10 or 11 wherein two or more of said PCM 1 marker SNPs are assessed.

13. A method as claimed in any of claims 4 to 12 wherein the 8p21-22 region in assessed by determining the binding of an oligonucleotide probe to the nucleic acid sample under conditions favourable for the specific annealing of these reagents to their complementary sequences.

14. A method as claimed in claim 13 wherein the probe comprises all or part of (i) the PCM1 sequence shown in any one of FIGS. 1-5, or (ii) a polymorphic form of the PCM1 sequence shown in any one of FIGS. 1-5, or (iii) the complement of either.

15. A method as claimed in claim 13 or claim 14 wherein the probe comprises a nucleic acid sequence which binds under stringent conditions specifically to one particular allele of a PCM1 marker and does not bind specifically to other alleles of the PCM 1 marker.

16. A method as claimed in any one of claims 13 to 15 wherein the probe is labelled and binding of the probe is determined by presence of the label.

17. A method as claimed in any of claims 4 to 16 wherein the method comprises amplifying a region of the 8p21-22 region comprising at least one PCM1 marker.

18. A method as claimed in claim 17 wherein the PCM1 gene is amplified.

19. A method as claimed in claim 17 wherein the region of the PCM1 gene which is amplified is within the intronic sequence 3′ to exon 4; in exon 4; or in the intronic sequence 5′ of exon 5.

20. A method as claimed in any one of claims 17 to 19 wherein a region of the 8p-22 region is amplified by use of two oligonucleotide primers.

21. A method as claimed in any one of claims 4 to 12 wherein the PCM 1 marker is assessed by a method selected from the group consisting of: strand conformation polymorphic marker analysis; heteroduplex analysis; RFLP analysis.

22. A method as claimed in claim 3 wherein the level of mRNA expression of the PCM1 gene is determined by Northern analysis or reverse transcriptase PCR.

23. A method of determining the presence or absence in a test sample of a PCM1 marker which is an SNP selected from the group consisting of the following positions numbered in accordance with the sequence of clone AB020866 as set out FIGS. 2-5:
(i) 80254,
(ii) 80123,
(iii) 87366,
(iv) 87507,
which method comprises determining the binding of an oligonucleotide probe to the nucleic acid sample, wherein the probe comprises all or part of (i) the PCM1 sequence shown in any one of FIGS. 2-5, or (ii) a polymorphic form of the PCM1 sequence shown in any one of FIGS. 2-5, or (iii) the complement of either.

24. A method of determining the presence or absence in a test sample of a PCM1 marker which is an SNP selected from the group consisting of the following positions numbered in accordance with the sequence of clone AB020866 as set out FIGS. 2-5:
(i) 80254,
(ii) 80123,
(iii) 87366,
(iv) 87507,
which method comprises use of two oligonucleotide primers capable of amplifying a portion of the PCM1 sequence which portion comprises at least one of said SNPs.

25. A method as claimed in any one of the preceding claims 4 to 21 wherein the PCM1 marker is assessed or confirmed by nucleotide sequencing.

26. An oligonucleotide probe for use in a method of any one of claims 13 to 16 or claim 23, which includes a PCM1 marker which is a microsatellite repeat present in the NT_{—}000501 contig on chromosome 8p21.3-22: D8S2612; D8S2613; D8S2614; D8S2615; D8S2616; D8S2617; or D8S2618, or which an SNP selected from the group consisting of the following positions numbered in accordance with the sequence of clone AB020866 as set out FIGS. 2-5:
(i) 80254,
(ii) 80123,
(iii) 87366,
(iv) 87507.

27. An oligonucleotide probe as claimed in claim 26 which probe comprises all or part of (i) the PCM1 sequence shown in any one of FIGS. 2-5, or (ii) a polymorphic form of the PCM1 sequence shown in any one of FIGS. 2-5, or (iii) the complement of either,

28. A PCR primer pair for use in a method of any one of claims 17 to 20 or claim 24 which primer pair comprises first and second primers which hybridise to DNA in regions including or flanking the PCM1 marker.

29. A primer pair as claimed in claim 28 wherein at least one of said primers is selected from the list consisting of:
(SEQ ID NO: 102)
5′-GGATAACAATTTCACACAGG-TGAGCCATTGATTATG-3′
(SEQ ID NO: 103)
5′-CACGACGTTGTAAAACGAC-AGTTGTCCCTGCAACCT-3′
(SEQ ID NO: 100)
5′-GGATAACAATTTCACACAGG-CCAAGTGTCTTTGGTTATCTTCG-3′
(SEQ ID NO: 101)
5′-CACGACGTTGTAAAACGAC-AGTCCGAACATCCTCCTCCT-3′

30. A primer pair as claimed in claim 28 wherein at least one of said primers is selected from the list consisting of:

| | |
|---|---|
| 5′AAT TCC CCA AAC AAA ACA ACA3′ | (SEQ ID NO: 85) |
| 5′AGG CTA TCC TTT CCT CAG CA3′ | (SEQ ID NO: 86) |
| 5′ATG TTC AGC CAC CAT CGT CT3′ | (SEQ ID NO: 87) |
| 5′CAG TGT CGC TGG AAA GTT GA3′ | (SEQ ID NO: 88) |
| 5′TCC CGA AGT GCT AGG ATT ACA3′ | (SEQ ID NO: 89) |
| 5′GCT CAG CAG GAA GAG GAA TG3′ | (SEQ ID NO: 90) |
| 5′AGA GGC CAG GCA CAA AAG TA3′ | (SEQ ID NO: 91) |
| 5′AAC ATT CCA GCA TCC CAA AG3′ | (SEQ ID NO: 92) |
| 5′GAC CCA CTG CCA CAC TCT TT3′ | (SEQ ID NO: 93) |
| 5′GGA GTG CGG CAT GAA ATT AT3′ | (SEQ ID NO: 94) |
| 5′ATA TGT ATA CAA TGT GTA TCT GTA | (SEQ ID NO: 95) |
| TC3′ | |
| 5′CCT TTT AGT TCC CAT TCC CAT T3′ | (SEQ ID NO: 96) |
| 5′TGA TGC AGG AGA ATT GCT TG3′ | (SEQ ID NO: 97) |
| 5′CCT ACT TGG CTG GGA TTC TG3′ | (SEQ ID NO: 98) |

31. A kit for determining the susceptibility of an individual to schizophrenia, or a method of diagnosis or prognosis of schizophrenia, the kit comprising a probe and\or primer of any one of claims 26 to 30.

32. A method of schizophrenia therapy, which method including the step of screening an individual for a genetic predisposition to schizophrenia in accordance with the method of any one of claims 1 to 22, whereby the predisposition is correlated with a PCM1 marker, and if a predisposition is identified, providing therapeutic treatment for the individual.

33. A method for identifying or isolating genetic loci associated with susceptibility to schizophrenia comprising screening genomic libraries with genetic sequence derived from PCM1 polymorphic markers located in the chromosomal region 8p21.3 and identifying open reading frames in regions adjacent to said genetic sequence.

34. A method for mapping polymorphic markers which are associated with susceptibility to schizophrenia, the method comprising identifying polymorphic markers which are in linkage disequilibrium with a PCM1 marker which is a microsatellite repeat present in the NT_{—}000501 contig on chromosome 8p21.3-22: D8S261; D8S2612; D8S2613; D8S2614; D8S2615; D8S2616; D8S2617; or D8S2618, or which an SNP selected from the group consisting of the following positions numbered in accordance with the sequence of clone AB020866 as set out FIGS. 2-5:
(i) 80254,
(ii) 80123,
(iii) 87366,
(iv) 87507.

35. A method of identifying a molecule for use in the diagnosis, prognosis or treatment of schizophrenia, which method comprises: admixing a test substance with a polypeptide encoded by a nucleic acid molecule comprising the PCM1 gene or a gene located within 1000 kb of the PCM1 locus and in linkage disequilibrium therewith; and measuring the level of activity of the polypeptide.

36. A method of identifying a molecule for use in the diagnosis, prognosis or treatment of schizophrenia, which method comprises: admixing a test substance with a polypeptide encoded by a nucleic acid molecule comprising the PCM1 gene or a gene located within 1000 kb of the PCM1 locus and in linkage disequilibrium therewith; and determining the binding of the test substance to the polypeptide.

37. An antibody specific for a polypeptide encoded by the PCM1 gene for use as a diagnostic and prognostic for schizophrenia.

38. A nucleic acid molecule comprising the PCM1 gene or a gene located within 1000 kb of the PCM1 locus and in linkage disequilibrium therewith for use in the treatment of schizophrenia.

39. A polypeptide encoded by the PCM1 gene or a gene located within 1000 kb of the PCM1 locus and in linkage disequilibrium therewith for use in the treatment of schizophrenia.

40. A method of treatment of schizophrenia comprising administering to a patient a substance which modulates expression from the PCM1 gene or a gene located within 1000 kb of the PCM1 locus and in linkage disequilibrium therewith, or administering a compound which modulates the level of activity of the PCM1 gene product or a gene located within 1000 kb of the PMC1 locus and in linkage disequilibrium therewith.
